(19) United States
(12) Patent Application Publication
(10) Pub. No.: US 2010/0226917 A1
(43) Pub. Date: Sep. 9, 2010

(54) METHODS FOR THE TREATMENT OF HEMATOLOGIC MALIGNANCIES

(75) Inventors: Jennifer Brown, Waltham, MA (US); Sonya Zabludoff, Waltham, MA (US)

Correspondence Address: ASTRAZENECA R&D BOSTON, 35 GATEHOUSE DRIVE, WALTHAM, MA, 02451-1215, US

(73) Assignee: AstraZeneca AB, Sodertalje (SE)

(21) Appl. No.: 12/597,133

(22) PCT Filed: Apr. 24, 2008
(86) PCT No.: PCT/GB08/50288

§ 371 Date: Apr. 1, 2010

Related U.S. Application Data
(60) Provisional application No. 60/914,453, filed on Apr. 27, 2007.

(51) Int. Cl. A61K 39/395

(52) U.S. Cl. 424/133.1; 424/130.1

(57) ABSTRACT

The present invention discloses, in part, therapies for treating hematologic malignancies, including B cell lymphomas and leukemias or B cell related tumors comprising the administration of a CHK1 inhibitor in combination with a B cell depleting antibody. The present invention further includes treating hematologic malignancies, including B cell lymphomas and leukemias, or B cell related tumors, which are resistant to cancer treatment comprising the administration of a CHK1 inhibitor.

### TECHNICAL FIELD

The present invention discloses therapies for treating hematologic malignancies, including B cell lymphomas and leukemias, or B cell related tumors.

### BACKGROUND OF THE INVENTION

Chemotherapy and radiation exposure are currently the major options for the treatment of cancer, but the therapeutic utility of both these approaches is severely limited by drastic adverse effects on normal tissue, and the frequent development of tumor cell resistance. It is therefore highly desirable to improve the efficacy of cancer treatments in a way that does not increase the toxicity associated with them. In some cases, one way to achieve enhanced efficacy is by employing anticancer agents in combination, wherein said combination causes a better therapeutic effect than that seen with each drug alone.

Combined treatment regimens would add to the therapies available to patients suffering from B cell related tumors or hematologic malignancies, including lymphoma patients, and might potentially even decrease the rate of relapse or overcome the resistance to a particular anticancer agent sometime seen in these patients. For example, in one possible scenario, a drug may act to increase the sensitivity of the malignant cell (e.g., B lymphoma cell) to the other drug of a combination therapy. In other scenarios, combinations of anticancer agents may have additive, or even synergistic, therapeutic effects.

One particular therapeutic agent used to treat hematologic malignancies is RITUXIN® (rituximab) (IDEC Pharmaceuticals Corporation, Cambridge, Mass.; Genentech, South San Francisco, Calif.). Rituximab is one of a new generation of monoclonal antibodies developed for the treatment of B cell lymphomas, and in particular, non-Hodgkin's lymphoma. Rituximab is a genetically engineered anti-CD20 monoclonal antibody with murine light- and heavy-chain variable regions and human gamma 1 heavy-chain and kappa light-chain constant regions. Rituximab acts by binding to the CD20 antigen on B cells which results in the lysis of the B cell by a mechanism thought to involve complement-dependent cytotoxicity (CDC) and antibody-dependent cell mediated cytotoxicity (ADCC). Combined immunochemotherapy using rituximab and the DNA damaging agent, fludarabine, has shown some promise in patients with chronic lymphocytic leukemia (CLL) (Schultz et al., Blood, Nov. 1, 2002 100(9):3115-3120).

DNA damaging agents like fludarabine have also been proposed for use in combination with inhibitors of checkpoint 1 kinase, (CHK1 inhibitors) which is an important regulatory component in the cell cycle. (See, for ex., Prudhomme, Recent Patents on Anti-Cancer Drug Discovery, 2006, 1:55). An individual cell replicates by making an exact copy of its chromosomes, and then segregating these into separate cells. This cycle of DNA replication, chromosome separation and division is regulated by mechanisms within the cell that maintain the order of the steps and ensure that each step is precisely carried out. Key to these processes are the cell cycle checkpoints (Hartwell et al., Science, Nov. 3, 1989, 246(4930):629-34) where cells may arrest to ensure DNA repair mechanisms have time to operate prior to continuing through the cycle into mitosis. There are two such checkpoints in the cell cycle—the G1/S checkpoint that is regulated by p53 and the G2/M checkpoint that is monitored by the Ser/Thr kinase checkpoint kinase 1 (CHK1). As the cell cycle arrest induced by these checkpoints is a crucial mechanism by which cells can overcome the damage resulting from radio- or chemotherapy, their abrogation by novel agents should increase the sensitivity of tumor cells to DNA damaging therapies. One approach to the design of compounds that abrogate the G2/M checkpoint is to develop inhibitors of the key G2/M regulatory kinase CHK1, and this approach has been shown to work in a number of proof of concept studies. (Koniaras et al., Oncogene, 2001, 20:7453; Luo et al., Neoplasia, 2001, 3:411; Busby et al., Cancer Res., 2000, 60:2108; Jackson et al., Cancer Res., 2000, 60:566).

Several CHK1 inhibitors have been identified. These compounds include aminopyrazoles, indazoles, tricyclic compounds, ureas, carbamates, diazepinones, pyrimidines, benzimidazole quinolones and macrocyclic compounds. (See, e.g., Prudhomme, Michelle, Novel checkpoint 1 inhibitors, Recent Patents on Anti-Cancer Drug Discovery (2006), 1(1), 55-68; Tao, Zhi-Fu; Lin, Nan-Horng, Chk1 inhibitors for novel cancer treatment, Anti-Cancer Agents in Medicinal Chemistry (2006), 6(4), 377-388; Kawabe, Takumi, G2 checkpoint abrogators as anticancer drugs, Molecular Cancer Therapeutics (2004), 3(4), 513-519; Prudhomme, Michelle, Combining DNA damaging agents and checkpoint 1 inhibitors, Current Medicinal Chemistry: Anti-Cancer Agents (2004), 4(5), 435-438). 2-ureidothiophene compounds and 3-ureidothiophene compounds are described as CHK1 inhibitors in WO03029241 and WO03028731, respectively. In addition, fused triazolones are described as CHK1 inhibitors in WO2004/081008. CHK1 inhibitors also include the thiophene carboxamides disclosed in WO2005/016909; the thiophene carboxamides disclosed in WO 2005/066163; and the substituted heterocycles, described in WO2006/106326.

Thus, it is contemplated herein that a combination of CHK1 inhibitors and B cell depleting antibodies would be useful to treat hematologic malignancies or other B cell related tumors. It is also contemplated herein that CHK1 inhibitors in combination with B cell depleting antibodies may be useful to treat patients suffering from hematologic disease or other B cell related tumors wherein said disease is resistant to cancer treatment, e.g., DNA damaging agents, including, but not limited to, fludarabine.

It is further contemplated herein that CHK1 inhibitors may be useful to treat patients suffering from hematologic disease or other B cell related tumors wherein said disease is resistant to cancer treatment, e.g., DNA damaging agents, including, but not limited to, fludarabine.

### SUMMARY OF THE INVENTION

In a first aspect the present invention provides a method to treat a patient suffering from a hematologic malignancy comprising administering to said patient a therapeutically effective amount of a CHK1 inhibitor simultaneously with or consecutively with (e.g., before or after) a B cell depleting antibody. In a related aspect, the hematologic malignancy is chronic lymphocytic leukemia (CLL). In another related aspect the B cell depleting antibody is an anti-CD20 antibody. In another related aspect, the anti-CD20 antibody is rituximab.

In another aspect, the present invention provides a method to treat a patient suffering from a B cell related tumor comprising administering to said patient a therapeutically effective amount of a CHK1 inhibitor simultaneously with or consecutively with (e.g. before or after) a B cell depleting antibody. In a related aspect the B cell depleting antibody is an anti-CD20 antibody. In another related aspect, the anti-CD20 antibody is rituximab.

In a further aspect, the present invention provides a method to treat a patient suffering from a hematologic malignancy or B cell related tumor that is resistant to cancer treatment, including treatment with a DNA damaging agent, comprising administering to said patient a therapeutically effective amount of a CHK1 inhibitor simultaneously with or consecutively with (e.g. before or after) a B cell depleting antibody. In a related aspect, the hematologic malignancy is chronic lymphocytic leukemia (CLL). In another related aspect the B cell depleting antibody is an anti-CD20 antibody. In another related aspect, the anti-CD20 antibody is rituximab. In another related aspect, the DNA damaging agent is fludarabine.

In a still further aspect, the present invention provides a method to treat a patient suffering from a hematologic malignancy or B cell related tumor that is resistant to cancer treatment, including treatment with a DNA damaging agent, comprising administering to said patient a therapeutically effective amount of a CHK1 inhibitor as a single agent. In a related aspect, the hematologic malignancy is CLL or AML. In another related aspect, the DNA damaging agent is fludarabine.

### BRIEF DESCRIPTION OF THE DRAWINGS

FIGS. 1(a-q) show line graphs of results from AML patient samples treated with a CHK1 inhibitor (▪), cytarabine (◯), or the combination of a CHK1 inhibitor and cytarabine ().

### DETAILED DESCRIPTION OF THE INVENTION

As used herein, “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, maleic, tartaric, citric, ascorbic, palmitic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic and isethionic. Pharmaceutically acceptable salts can be synthesized from the parent compound that contains a basic or acidic moiety by conventional chemical methods.

With regard to the administration of a compound, antibody or other drug substance, as used herein the term “consecutively with” refers to before or after the administration of another compound, antibody or other drug substance.

A “B cell surface marker” or “B cell target” or “B cell antigen” herein is an antigen expressed on the surface of a B cell which can be targeted with an antagonist which binds thereto. Exemplary B cell surface markers include the CD10, CD19, CD20, CD21, CD22, CD23, CD24, CD37, CD53, CD72, CD73, CD74, CDw75, CDw76, CD77, CDw78, CD79a, CD79b, CD80, CD81, CD82, CD83, CDw84, CD85 and CD86 leukocyte surface markers. The B cell surface marker of particular interest is preferentially expressed on B cells compared to other non-B cell tissues of a mammal and may be expressed on both precursor B cells and mature B cells. In one embodiment, the marker is one like CD20 or CD19, which is found on B cells throughout differentiation of the lineage from the stem cell stage up to a point just prior to terminal differentiation into plasma cells.

A “CD20” antigen is a 35 kDa, non-glycosylated phosphoprotein found on the surface of greater than 90% of B cells from peripheral blood or lymphoid organs. CD20 is expressed during early pre-B cell development and remains until plasma cell differentiation. CD20 is present on both normal B cells as well as malignant B cells. Other names for CD20 in the literature include “B-lymphocyte-restricted antigen” and “Bp35”. The CD20 antigen is described in, e.g., Clark et al. PNAS (USA) 82:1766 (1985).

“B cell depleting antibodies” are defined as those antibodies which bind to receptors or other targets on the surface of hematologic malignant cells, e.g., tumorigenic B cells, and mediate their destruction or depletion when they bind, e.g., by inducing apoptosis. Such antibodies include, but are not limited to, anti-CD20, anti-CD19, anti-CD22, anti-CD 21, anti-CD23, anti-CD28, anti-CD37, anti-CD40, anti-CD52 antibodies. An example of an anti-CD20 antibody is RITUXIN® (rituximab). B cell depleting antibodies also include antibodies that destroy B cells via other mechanisms. For example, these include radiolabeled antibodies that facilitate the destruction of tumor cells by binding to the B cell surface and delivering a lethal dose of radiation. These include 131 I-Lym-1 (anti-HLA-D), 131 I-tositumomab (BEXXAR®), ibritumomab tiuxetan (Y-90, In-111 ZEVALIN®) and 90 Y-epratuzumab. Such antibodies, as well as antibodies conjugated to toxins, may also be used in conjunction with the CHK1 inhibitors disclosed herein and will be familiar to one of skill in the art.

A “CHK1 inhibitor” refers to any compound or substance that can inhibit the activity of checkpoint 1 kinase and/or checkpoint 2 kinase. The CHK1 inhibitors useful in the present invention can be any of those known in the art, e.g. those mentioned above e.g., those described in Prudhomme, Michelle, Novel checkpoint 1 inhibitors, Recent Patents on Anti-Cancer Drug Discovery (2006), 1(1), 55-68; Tao, Zhi-Fu; Lin, Nan-Horng, Chk1 inhibitors for novel cancer treatment, Anti-Cancer Agents in Medicinal Chemistry (2006), 6(4), 377-388; Kawabe, Takumi, G2 checkpoint abrogators as anticancer drugs, Molecular Cancer Therapeutics (2004), 3(4), 513-519; Prudhomme, Michelle, Combining DNA damaging agents and checkpoint 1 inhibitors, Current Medicinal Chemistry: Anti-Cancer Agents (2004), 4(5), 435-438; the 2-ureidothiophene compounds and 3-ureidothiophene compounds described in WO03029241 and WO03028731, respectively; the fused triazolones described as CHK1 inhibitors in WO2004/081008; the thiophene carboxamides disclosed in WO2005/016909; the thiophene carboxamides disclosed in WO 2005/066163; and the substituted heterocycles, described in WO2006/106326.

CHK1 inhibitors may exist in particular geometric or stereoisomeric forms. The present invention takes into account all such compounds, including cis- and trans isomers, R- and S-enantiomers, diastereomers, (D)-isomers, (L)-isomers, the racemic mixtures thereof, and other mixtures thereof. Also, the CHK1 inhibitors discussed herein may exist in free or salt form, e.g., as acid addition salts. In this specification, unless otherwise indicated, it is understood that the compounds disclosed herein include the compounds in any form, e.g., free or acid addition salt form, or where the compounds contain acidic substituents, in base addition salt form. As the compounds disclosed herein are intended for use as pharmaceuticals, pharmaceutically acceptable salts are preferred.

A “DNA damaging agent” refers to those compounds or substances that damage DNA such that a cell is killed or prevented from growing. DNA damaging agents include, but are not limited to, chlorambucil, cyclophosphamide, melphalan, carboplatinum, daunorubicin, doxorubicin, idarubicin, and mitoxantrone, as well as methotrexate, fludarabine, and cytarabine.

A “hematologic malignancy” includes any malignancy associated with cells in the bloodstream; bone marrow; and the lymphoid system including in the liver, spleen, and lymph nodes. Examples thereof include B and T cell lymphomas, leukemias including, but not limited to, low grade/follicular non-Hodgkin's lymphoma (NHL), small lymphocytic (SL) NHL, T or B prolymphocytic leukemia, follicular NHL, diffuse large B cell NHL, peripheral T cell lymphomas, mantle cell lymphoma, marginal zone lymphomas, B or T cell lymphoblastic lymphoma, Burkitt's lymphoma, Waldenstrom's Macroglobulinemia or lymphoplasmacytic lymphoma, chronic leukocytic leukemia, acute myelogenous leukemia (AML), acute lymphoblastic leukemia, chronic lymphocytic leukemia (CLL), chronic myelogenous leukemia, lymphoblastic leukemia, multiple myeloma, lymphocytic leukemia, monocytic leukemia, myelogenous leukemia, and promyelocytic leukemia. It should be clear to those of skill in the art that these pathological conditions may often have different names due to differing/changing classification systems.

A “B cell related tumor” refers herein to a solid, non-hematologic (non-lymphoid) tumor, i.e., a non-hematologic malignancy having B cell involvement where B cells are involved in a “protumor” response. For example, B cells are somehow involved in impeding the body's immune defense system against such malignancy by somehow promoting or maintaining the tumorigenic state. As such, B cells are involved, but are not themselves the cancerous cells. With respect thereto, WO 020864 A1, incorporated by reference herein, describes the treatment of solid, non-lymphoid tumors using antibodies that target B cells, including rituximab. It was reported therein that this treatment resulted in pronounced anti-tumor responses, even in patients with advanced colorectal cancer, lung cancer and liver cancer. B cell related solid tumors may be palpable tumors, typically at least 0.5 mm in diameter, more typically at least 1.0 mm in diameter. Examples thereof include colorectal cancer, liver cancer, breast cancer, lung cancer, head and neck cancer, stomach cancer, testicular cancer, prostate cancer, ovarian cancer, uterine cancer and others. These cancers may be in the early stages (precancer), intermediate (Stages I and II) or advanced, including solid tumors that have metastasized. These solid tumors will preferably be cancers wherein B cells elicit a protumor response, i.e. the presence of B cells is involved in tumor development, maintenance or metastasis.

A used herein, a hematologic disease or other B cell related tumor that is “resistant to” cancer treatment refers to a disease that is refractory or resistant to treatment with other anticancer agents, including, but not limited to, treatment with DNA damaging agents, e.g. fludarabine. The disease may, at one time, have responded to the other cancer agents, e.g. DNA damaging agents, but is now resistant to such treatment.

A “B cell antagonist” is a molecule which, upon binding to a B cell surface marker, destroys or depletes B cells in a mammal and/or interferes with one or more B cell functions, e.g. by reducing or preventing a humoral response elicited by the B cell. The antagonist preferably is able to deplete B cells (i.e. reduce circulating B cell levels) in a mammal treated therewith. Such depletion may be achieved via various mechanisms such antibody-dependent cell mediated cytotoxicity (ADCC) and/or complement dependent cytotoxicity (CDC), inhibition of B cell proliferation and/or induction of B cell death (e.g. via apoptosis). Antagonists included within the scope of the present invention include antibodies, synthetic or native sequence peptides and small molecule antagonists which bind to the B cell marker, optionally conjugated with or fused to a cytotoxic agent. The preferred antagonist comprises an antibody, more preferably a B cell depleting antibody.

“Antibody-dependent cell-mediated cytotoxicity” and “ADCC” refer to a cell-mediated reaction in which nonspecific cytotoxic cells that express Fc receptors (FcRs) (e.g. Natural Killer (NK) cells, neutrophils, and macrophages) recognize bound antibody on a target cell and subsequently cause lysis of the target cell. To assess ADCC activity of a molecule of interest, an in vitro ADCC assay, such as that described in U.S. Pat. No. 5,500,362 or U.S. Pat. No. 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. PNAS (USA) 95:652-656 (1998).

“Complement dependent cytotoxicity” or “CDC” refers to the ability of a molecule to lyse a target in the presence of complement. The complement activation pathway is initiated by the binding of the first component of the complement system (Clq) to a molecule (e.g. an antibody) complexed with a cognate antigen. To assess complement activation, a CDC assay, e.g. as described in Gazzano-Santoro et al., J. Immunol. Methods 202:163 (1996), may be performed.

The term “antibody” herein is used in the broadest sense and specifically covers intact monoclonal antibodies, polyclonal antibodies, multispecific antibodies (e.g. bispecific antibodies) formed from at least two intact antibodies, and antibody fragments so long as they exhibit the desired biological activity. Antibodies may be produced by one of skill in the art using conventional methods.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations, which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature, 256:495 (1975), or may be made by recombinant DNA methods (see, e.g., U.S. Pat. No. 4,816,567). The “monoclonal antibodies” may also be isolated from phage antibody libraries using the techniques described in Clackson et al., Nature, 352:624-628 (1991) and Marks et al., J. Mol. Biol., 222:581-597 (1991), for example. The monoclonal antibodies herein specifically include but are not limited to “chimeric” or “humanized” forms.

Examples of antibodies which bind the CD20 antigen include: “C2B8” which is “rituximab” (“RITUXAN®”) (U.S. Pat. No. 5,736,137, expressly incorporated herein by reference); the yttrium-[90]-labeled 2138 murine antibody designated “Y2B8” (U.S. Pat. No. 5,736,137, expressly incorporated herein by reference); murine IgG2a “131” optionally labeled with 1311 to generate the “1311-B1” antibody (BEXXARTM®) (U.S. Pat. No. 5,595,721, expressly incorporated herein by reference); murine monoclonal antibody “1F5” (Press et al. Blood 69(2): 584-591 (1987)); “chimeric 2H7” antibody (U.S. Pat. No. 5,677,180 expressly incorporated herein by reference); and monoclonal antibodies L27, G28-2, 93-1133, B-C1 or NU-B2 available from the International Leukocyte Typing Workshop (Valentine et al., In: Leukocyte Typing III (McMichael, Ed., p. 440, Oxford University Press (1987)).

The terms “rituximab” or “RITUXAN®” herein refer to the genetically engineered chimeric murine/human monoclonal antibody directed against the CD20 antigen and designated “C2B8” in U.S. Pat. No. 5,736,137, expressly incorporated herein by reference. The antibody is an IgG, kappa immunoglobulin containing murine light and heavy chain variable region sequences and human constant region sequences. Rituximab has a binding affinity for the CD20 antigen of approximately 8.0 nM. It is commercially available, e.g. from Genentech (South San Francisco, Calif.).

The words “treat”, “treatment” and “treating” are to be understood as embracing prophylaxis and treatment or amelioration of symptoms of disease as well as treatment of the cause of the disease. Those in need of treatment include those already with a disease or disorder to be treated as well as those in which the disease or disorder is to be prevented, patients which show resistance to treatment or those prone to relapse. Hence, the patient may have been diagnosed as having the disease or disorder or may be predisposed or susceptible to the disease or to relapse or is resistant to treatment.

The expression “therapeutically effective amount” refers to an amount of a drug substance (e.g., inhibitory compounds disclosed herein and/or B cell depleting antibody) effective for treatment or prophylaxis or amelioration of symptoms of a hematologic malignancy or other B cell related tumor discussed herein.

As discussed above, it is contemplated herein that CHK1 inhibitors, including those compounds disclosed in detail herein, have utility for the treatment of hematologic malignancies and other B cell related tumors, including those that may be resistant to DNA damaging agents or other forms of cancer treatment, in combination therapies with B cell depleting antibodies, particularly a B cell depleting anti-CD20 antibody, particularly rituximab, according to the methods of the invention described in detail herein below.

The methods disclosed herein can be administered to a patient prior to, concurrently with or after administration of, at least one other chemotherapeutic agent (e.g. triple and even multiple combinations of agents are contemplated herein) and may be useful for avoiding, decreasing or overcoming the resistance to chemotherapeutic agents often exhibited by malignant cells. Patients include those who may have relapsed following chemotherapy or other cancer treatment or whose malignancy or tumor is resistant to chemotherapy or other cancer treatment. In fact, because resistance of B cells is often apparent only after a patient has relapsed following, or is resistant to, a first treatment with a therapeutic agent, the methods of the present invention will often encompass treating patients with hematologic malignancies such as B cell lymphoma or leukemia who have relapsed following, or have demonstrated resistance to, chemotherapy or other cancer treatment. For example, resistance to fludarabine is frequently seen in those suffering from CLL and it is contemplated herein that the methods of the present invention would be useful to treat such individuals. In addition, the combination therapies disclosed herein may also be used in conjunction with other therapies or prior to or after other therapies in patients newly diagnosed with a hematologic malignancy to decrease the chance of relapse, and increase the length and duration of the response to therapy.

The methods of the present invention are appropriate to treat a wide variety of hematologic malignancies, especially B cell lymphomas and leukemias, including but not limited to, low grade/follicular non-Hodgkin's lymphoma (NHL), small lymphocytic (SL) NHL, intermediate grade/follicular NHL, intermediate grade diffuse NHL, high grade immunoblastic NHL, high grade lymphoblastic NHL, high grade small non-cleaved cell NHL, bulky disease NHL and Waldenstrom's Macroglobulinemia, chronic leukocytic leukemia, acute myelogenous leukemia, acute lymphoblastic leukemia, chronic lymphocytic leukemia, chronic myelogenous leukemia, lymphoblastic leukemia, lymphocytic leukemia, monocytic leukemia, myelogenous leukemia, and promyelocytic leukemia. As discussed above, one of skill in the art is aware that these lymphomas may be known by different names, e.g., given the existence of different classification systems (e.g., based on cytology or level of “aggressiveness” of a particular malignancy). As previously discussed herein, a particular disease to be targeted is chronic lymphocytic leukemia (CLL).

The methods of the present invention include methods to treat B cell related tumors such as solid, non-lymphoid tumors. In this method a CHK1 inhibitor and a B cell depleting antibody, e.g., rituximab, may be administered simultaneously or consecutively (e.g. before or after) to the solid tumor site, e.g., by injection proximate to or directly at the tumor site, e.g., by intravenous injection at a vein proximate to the tumor.

Also included in the present invention are kits for accomplishing the disclosed methods. A kit according to the present invention comprises at least one CHK1 inhibitor and at least one B cell depleting antibody, which may be readily admixed or resuspended with a pharmaceutically acceptable carrier and conveniently injected into a patient.

The compounds disclosed herein may be administered in different ways, all of which are familiar to one of skill in the art, e.g., sublingually, intramuscularly, subcutaneously, topically, intranasally, intraperitoneally, intrathoracially, intravenously, epidurally, intrathecally, intracerebroventricularly, injection into the joints, or orally where appropriate (typically contraindicated for administration of antibodies).

Dosages will depend on the route of administration, the severity of the disease, age and weight of the patient and other factors normally considered by the attending physician, when determining the individual regimen and dosage level as the most appropriate for a particular patient. In addition, the pharmaceutical compositions can be in unit dosage form. In such form, the composition is divided into unit doses containing appropriate quantities of the active component. The unit dosage form can be a packaged preparation, the package containing discrete quantities of the preparations, for example, packeted tablets, capsules, and powders in vials or ampoules. The unit dosage form can also be a capsule, cachet, or tablet itself, or it can be the appropriate number of any of these packaged forms.

In general, with regard to CHK1 inhibitors, including those specifically disclosed herein, satisfactory results, e.g., for the treatment of diseases as hereinbefore set forth are indicated to be obtained at dosages of the order from about 0.01 to 2.0 mg/kg. In larger mammals, for example humans, an indicated daily dosage may accordingly be in the range of from about 0.75 to 1000 mg.

Depending on the patient and extent of disease, the anti-B cell target binding antibody (e.g., B cell depleting antibody), e.g., rituximab, may be administered at a dosage ranging from 0.01 to about 100 mg/kg, more preferably from about 0.1 to 50 mg/kg, and most preferably from about 0.4 to 20 mg/kg of body weight. Effective dosages may be lower in combined therapeutic regimens with CHK1 inhibitors, because the proliferative potential of B lymphoma cells may be reduced. Effective doses of B cell depleting antibody will be apparent to one of skill in the art, depending on the CHK1 inhibitor utilized in the combination therapy and amount thereof.

For preparing pharmaceutical compositions from the compounds and antibodies disclosed herein, inert, pharmaceutically acceptable carriers can be either solid or liquid. Solid form preparations include powders, tablets, dispersible granules, capsules, cachets, and suppositories. The term “composition” is intended to include the formulation of an active component(s) with encapsulating material as a carrier providing a capsule in which the active component (with or without other carriers) is surrounded by a carrier which is thus in association with it. Similarly, cachets are included. Tablets, powders, cachets, and capsules can be used as solid dosage forms suitable for oral administration.

A solid carrier can be one or more substances which may also act as diluents, flavoring agents, solubilizers, lubricants, suspending agents, binders, or tablet disintegrating agents; it can also be an encapsulating material.

In powders, the carrier may be a finely divided solid which is in a mixture with the finely divided active component. In tablets, the active component may be mixed with the carrier having the necessary binding properties in suitable proportions and compacted in the shape and size desired.

For preparing suppository compositions, a low-melting wax such as a mixture of fatty acid glycerides and cocoa butter is first melted and the active ingredient is dispersed therein by, for example, stirring. The molten homogeneous mixture is then poured into convenient sized molds and allowed to cool and solidify.

Suitable carriers include magnesium carbonate, magnesium stearate, talc, lactose, sugar, pectin, dextrin, starch, tragacanth, methyl cellulose, sodium carboxymethyl cellulose, a low-melting wax, cocoa butter, and the like.

Liquid form compositions include solutions, suspensions, and emulsions. Sterile water or water-propylene glycol solutions of the active components may be mentioned as an example of liquid preparations suitable for parenteral administration. Liquid compositions can also be formulated in solution in aqueous polyethylene glycol solution. Aqueous solutions for oral administration can be prepared by dissolving the active component in water and adding suitable colorants, flavoring agents, stabilizers, and thickening agents as desired. Aqueous suspensions for oral use can be made by dispersing the finely divided active component in water together with a viscous material such as natural synthetic gums, resins, methylcellulose, sodium carboxymethyl cellulose, and other suspending agents known in the pharmaceutical formulation art.

It is understood that the CHK1 inhibitors for use in the methods of the present invention include compounds in free form or in the form of a pharmaceutically acceptable salt of the compound or in the form of a pharmaceutically acceptable solvate of the compound or salt. Any CHK 1 inhibitors may be used in the instant invention, these include, e.g., any of the aforementioned CHK1 inhibitors. In particular, CHK1 inhibitors include the thiophene carboxamides disclosed in WO2005/066163. These CHK1 inhibitors can be prepared in a number of ways well known to one skilled in the art of organic synthesis, including, but not limited to, the methods of synthesis described in detail in WO 2005/066163, the entire contents of which are hereby incorporated by reference. Thiophene carboxamides of interest as CHK1 inhibitors include compounds of the aforementioned WO 2005/066163 as shown in Formula (I):

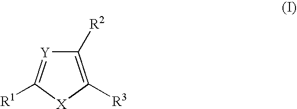

wherein:

X is selected from NH, S and O;

Y is selected from CH or N;

R¹ is selected from cyano, isocyano, C₁₋₆alkyl, —NR¹¹R¹², C₁₋₆alkoxy, C₂₋₆alkenyl, C₂₋₆alkynyl, cycloalkyl, cycloalkenyl, aryl, and heterocyclyl, provided R¹ is not thienyl; and wherein R¹ may be optionally substituted on one or more carbon atoms by one or more R⁹; and wherein if said R¹ contains an —NH— moiety, the nitrogen of said moiety may be optionally substituted by a group selected from R¹⁰;

R² and R³ are each independently selected from —C(═O)NR⁶R⁷, —SO₂NR¹⁶R¹⁷, —NHC(═O)NHR⁴, and —NHC(═NR⁸)NH₂;

R⁴ is selected from H, OH, —NR¹¹R¹², benzyl, C₁₋₆alkoxy, cycloalkyl, cycloalkenyl, aryl, heterocyclyl, mercapto, CHO, —COaryl, —CO(C₁₋₆alkyl), —CONR³⁰R³¹, —CO₂(C₁₋₆alkyl), —CO₂aryl, —CO₂NR³⁰R³¹, —Salkyl, —SO(C₁₋₆alkyl), —SO₂(C₁₋₆alkyl), —Saryl, —SOaryl, —SO₂aryl, —SO₂NR³⁰R³¹, and —(C₁₋₆alkyl)SO₂NR³⁰R³¹ wherein R⁴ may be optionally substituted on one or more carbon atoms by one or more R¹⁵; and wherein if said heterocyclyl contains a —NH— moiety, the nitrogen may be optionally substituted by a group selected from R¹⁴;

R⁶ and R⁷ are each independently selected from H, OH, OCH₃, C₁₋₆alkoxy, —NH₂, —NHCH₃, —N(CH₃)₂, (C₁₋₃alkyl)NR¹¹R¹², —CH₂CH₂OH, cycloalkyl, and a 5, 6, or 7-membered heterocyclyl ring containing at least one nitrogen atom, provided R⁶ and R⁷ are not both H; alternatively R⁶ and R⁷ taken together with the N to which they are attached form a heterocyclic ring; wherein R⁶ and R⁷ independently of each other may be optionally substituted on one or more carbon atoms by one or more R¹⁸; and wherein if said heterocyclyl contains a —NH— moiety, the nitrogen of said moiety may be optionally substituted by a group selected from R¹⁹;

R⁸ is selected from cyano, isocyano, —SO₂(C₁₋₆alkyl), —SO₂-aryl; —SO₂cycloalkyl, —SO₂cycloalkenyl, —SO₂heterocyclyl, and CF₃; wherein R⁸ may be optionally substituted on one or more carbon atoms by one or more R²³;

R⁹, R¹⁵, R¹⁸, R²³, R²⁴ and R³³ are each independently selected from halogen, nitro, —NR³⁰R³¹, cyano, isocyano, C₁₋₆alkyl, C₂₋₆alkenyl, C₂₋₆alkynyl, aryl, cycloalkyl, heterocyclyl, hydroxy, keto(═O), —O(C₁₋₆alkyl), —Oaryl, —OCOalkyl, —NHCHO, —N(C₁₋₆alkyl)CHO, —NHCONR³⁰R³¹, —N(C₁₋₆alkyl)CONR³⁰R³¹, —NHCOalkyl, —NHCO₂(C₁₋₆alkyl); —NHCO₂H, —N(C₁₋₆alkyl)CO(C₁₋₆alkyl), —NHSO₂(C₁₋₆alkyl), carboxy, -amidino, —CHO, —CONR³⁰R³¹, —CO(C₁₋₆alkyl), —COheterocyclyl, —COcycloalkyl, —CO₂H, —CO₂(C₁₋₆alkyl), —CO₂(aryl), —CO₂(NR³⁰R³¹), mercapto, —SO(C₁₋₆alkyl), —SO₂(C₁₋₆alkyl), —SO₂NR³⁰R³¹; wherein R⁹, R¹⁵, R¹⁸, R²³, R²⁴ and R³³ independently of each other may be optionally substituted on carbon by one or more R²⁰ and on nitrogen of any moiety that contains an NH or NH₂ by R²¹;

R¹⁰, R¹⁴, R¹⁹, R²⁵ and R³⁴ are each independently selected from halogen, nitro, —NR³⁰R³¹, cyano, isocyano, C₁₋₆alkyl, C₂₋₆alkenyl, C₂₋₆alkynyl, aryl, cycloalkyl, heterocyclyl, hydroxy, keto(═O), —O(C₁₋₆alkyl), —Oaryl, —OCOalkyl, —NHCHO, —N(C₁₋₆alkyl)CHO, —NHCONR³⁰R³¹, —N(C₁₋₆alkyl)CONR³⁰R³¹, —NHCOalkyl, —NHCO₂(C₁₋₆alkyl); —NHCO₂H, —N(C₁₋₆alkyl)CO(C₁₋₆alkyl), —NHSO₂(C₁₋₆alkyl), carboxy, -amidino, —CHO, —CONR³⁰R³¹, —CO(C₁₋₆alkyl), —COheterocyclyl, —COcycloalkyl, —CO₂H, —CO₂(C₁₋₆alkyl), —CO₂(aryl), —CO₂(NR³⁰R³¹), mercapto, —SO(C₁₋₆alkyl), —SO₂(C₁₋₆alkyl), —SO₂NR³⁰R³¹; wherein R¹⁰, R¹⁴, R¹⁹, R²⁵ and R³⁴ independently of each other may be optionally substituted on carbon by one or more R²² and on nitrogen of any moiety that contains an NH or NH₂ by R²³;

R¹¹ and R¹² are independently selected from H, C₁₋₆alkyl, cycloalkyl, aryl, heterocyclyl; alternatively R¹¹ and R¹² taken together with the N to which they are attached form a heterocyclic ring; wherein R¹¹ and R¹² independently of each other may be optionally substituted on carbon by one or more R³³, and wherein if said heterocyclyl contains a —NH— moiety, the nitrogen of said moiety may be optionally substituted by a group selected from R³⁴;

R¹⁶ and R¹⁷ are each independently selected from H, OH, OCH₃, C₁₋₆alkoxy, NH₂, —NHCH₃, —N(CH₃)₂, (C₁₋₃alkyl)NR¹¹R¹², —CH₂CH₂OH, cycloalkyl, aryl, or a 5, 6 or 7-membered heterocyclyl ring containing at least one nitrogen atom, provided R¹⁶ and R¹⁷ are not both H; alternatively R¹⁶ and R¹⁷ taken together with the N to which they are attached form an optionally substituted heterocyclic ring; wherein R¹⁶ and R¹⁷ independently of each other may be optionally substituted on one or more carbon atoms by one or more R²⁴; and wherein if said heterocyclyl contains an —NH— moiety, the nitrogen of said moiety may be optionally substituted by a group selected from R²⁵;

R²⁰, R²² and R³² are each independently selected from halogen, nitro, —NR³⁰R³¹, cyano, isocyano, C₁₋₆alkyl, C₂₋₆alkenyl, C₂₋₆alkynyl, aryl, cycloalkyl, heterocyclyl, hydroxy, keto(═O), —Oaryl, —OCOalkyl, —NHCHO, —N(C₁₋₆alkyl)CHO, —NHCONR³⁰R³¹, —N(C₁₋₆alkyl)CONR³⁰R³¹, —NHCOalkyl, —NHCO₂(C₁₋₆alkyl); —NHCO₂H, —N(C₁₋₆alkyl)CO(C₁₋₆alkyl), —NHSO₂(C₁₋₆alkyl), carboxy, -amidino, —CHO, —CONR³⁰R³¹, —CO(C₁₋₆alkyl), —COheterocyclyl, —COcycloalkyl, —CO₂H, —CO₂(C₁₋₆alkyl), —CO₂(aryl), —CO₂(NR³⁰R³¹), mercapto, —SO(C₁₋₆alkyl), —SO₂(C₁₋₆alkyl), —SO₂NR³⁰R³¹; wherein R²⁰, R²¹ and R³² independently of each other may be optionally substituted on carbon by one or more R²⁶ and on nitrogen of any moiety that contains an NH or NH₂ by R²⁷;

R²¹, R²³ and R³⁵ are each independently selected from halogen, nitro, —NR³⁰R³¹, cyano, isocyano, C₁₋₆alkyl, C₂₋₆alkenyl, C₂₋₆alkynyl, aryl, cycloalkyl, heterocyclyl, hydroxy, keto(═O), —Oaryl, —OCOalkyl, —NHCHO, —N(C₁₋₆alkyl)CHO, —NHCONR³⁰R³¹, —N(C₁₋₆alkyl)CONR³⁰R³¹, —NHCOalkyl, —NHCO₂(C₁₋₆alkyl); —NHCO₂H, —N(C₁₋₆alkyl)CO(C₁₋₆alkyl), —NHSO₂(C₁₋₆alkyl), carboxy, -amidino, —CHO, —CONR³⁰R³¹, —CO(C₁₋₆alkyl), —COheterocyclyl, —COcycloalkyl, —CO₂H, —CO₂(C₁₋₆alkyl), —CO₂(aryl), —CO₂ mercapto, —SO(C₁₋₆alkyl), —SO₂(C₁₋₆alkyl), —SO₂NR³⁰R³¹; wherein R²¹, R²³ and R³⁵ independently of each other may be optionally substituted on carbon by one or more R²⁸ and on nitrogen of any moiety that contains an NH by R²⁹;

R²⁶ and R²⁸ are each independently selected from halogen, nitro, —NR³⁰R³¹, cyano, isocyano, C₁₋₆alkyl, C₂₋₆alkenyl, C₂₋₆alkynyl, aryl, cycloalkyl, heterocyclyl, hydroxy, keto(═O), —O(C₁₋₆alkyl), —Oaryl, —OCOalkyl, —NHCHO, —N(C₁₋₆alkyl)CHO, —NHCONR³⁰R³¹, —N(C₁₋₆alkyl)CONR³⁰R³¹, —NHCOalkyl, —NHCO₂(C₁₋₆alkyl); —NHCO₂H, —N(C₁₋₆alkyl)CO(C₁₋₆alkyl), —NHSO₂(C₁₋₆alkyl), carboxy, -amidino, —CHO, —CONR³⁰R³¹, —CO(C₁₋₆alkyl), —COheterocyclyl, —COcycloalkyl, —CO₂H, —CO₂(C₁₋₆alkyl), —CO₂(aryl), —CO₂(NR³⁰R³¹), mercapto, —S(C₁₋₆alkyl), —SO(C₁₋₆alkyl), —SO₂(C₁₋₆alkyl), —SO₂NR³⁰R³¹;

R²⁷ and R²⁹ are each independently selected from halogen, nitro, —NR³⁰R³¹, cyano, isocyano, C₁₋₆alkyl, C₂₋₆alkenyl, C₂₋₆alkynyl, aryl, cycloalkyl, heterocyclyl, hydroxy, keto(═O), —O(C₁₋₆alkyl), —Oaryl, —OCOalkyl, —NHCHO, —N(C₁₋₆alkyl)CHO, —NHCONR³⁰R³¹, —N(C₁₋₆alkyl)CONR³⁰R³¹, —NHCOalkyl, —NHCO₂(C₁₋₆alkyl); —NHCO₂H, —N(C₁₋₆alkyl)CO(C₁₋₆alkyl), —NHSO₂(C₁₋₆alkyl), carboxy, -amidino, —CHO, —CONR³⁰R³¹, —CO(C₁₋₆alkyl), —COheterocyclyl, —COcycloalkyl, —CO₂H, —CO₂(C₁₋₆alkyl), —CO₂(aryl), —CO₂(NR³⁰R³¹), mercapto, —S(C₁₋₆alkyl), —SO(C₁₋₆alkyl), —SO₂(C₁₋₆alkyl), —SO₂NR³⁰R³¹;

R³⁰ and R³¹ are each independently selected from halogen, nitro, —NH₂, cyano, isocyano, C₁₋₆alkyl, C₂₋₆alkenyl, C₂₋₆alkynyl, aryl, cycloalkyl, heterocyclyl, hydroxy, keto(═O), —O(C₁₋₆alkyl), —Oaryl, —OCOalkyl, —NHCHO, —N(C₁₋₆alkyl)CHO, —NHCONR¹¹R¹², —N(C₁₋₆alkyl)CONR¹¹R¹², —NHCOalkyl, —NHCO₂(C₁₋₆alkyl); —NHCO₂H, —N(C₁₋₆alkyl)CO(C₁₋₆alkyl), —NHSO₂(C₁₋₆alkyl), carboxy, -amidino, —CHO, —CONR³⁰R³¹, —CO(C₁₋₆alkyl), —COheterocyclyl, —COcycloalkyl, —CO₂H, —CO₂(C₁₋₆alkyl), —CO₂(aryl), —CO₂(NR³⁰R³¹), mercapto, —S(C₁₋₆alkyl), —SO(C₁₋₆alkyl), —SO₂(C₁₋₆alkyl), —SO₂NR¹¹R¹²; wherein R³⁰ and R³¹ independently of each other may be optionally substituted on carbon by one or more R³²; and wherein if said heterocyclyl contains a —NH— or NH₂ moiety, the nitrogen of said moiety may be optionally substituted by a group selected from R³⁵;

or a pharmaceutically acceptable salt thereof;
provided that when X is S; Y is CH; R₂ is C(═O)NR⁶R⁷; and R³ is NHC(═O)NHR⁴; then R¹ cannot be

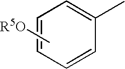

wherein R⁵ is selected from H, optionally substituted carbocyclyl, or optionally substituted C₁₋₆alkyl; with the further proviso that said compound is not
5-Methyl-2-ureido-thiophene-3-carboxylic acid (1-ethyl-piperidin-3-yl)-amide;
[3-((S)-3-Amino-azepane-1-carbonyl)-5-ethyl-thiophen-2-yl]-urea;
2-Morpholin-4-yl-4-ureido-thiazole-5-carboxylic acid (S)-piperidin-3-ylamide;
2-Methyl-5-ureido-oxazole-4-carboxylic acid (S)-piperidin-3-ylamide;
5-(4-Chloro-phenyl)-3-{3-[(R)-1-(2,2,2-trifluoro-acetyl)-piperidin-3-yl]-ureido}-thiophene-2-carboxylic acid (S)-piperidin-3-ylamide; or
N-(3-{[(3S)-3-aminoazepan-1-yl]carbonyl}-5-pyridin-2-yl-2-thienyl)urea.

Compounds of Formula (I) which are of particular interest include the following:
1.1: 5-(3-Fluoro-phenyl)-3-ureido-thiophene-2-carboxylic acid (S)-piperidin-3-ylamide;
1.2: 5-Phenyl-2-ureido-thiophene-3-carb oxylic acid (S)-piperidin-3-ylamide;
1.3: 5-(3,5-Difluoro-phenyl)-2-ureido-thiophene-3-carboxylic acid (S)-piperidin-3-ylamide;
1.4: 5-(4-Fluoro-phenyl)-2-ureido-thiophene-3-carb oxylic acid (S)-piperidin-3-ylamide;
1.5: 5-(4-Chloro-phenyl)-2-ureido-thiophene-3-carboxylic acid (S)-piperidin-3-ylamide;
1.6: 5-(3-Chloro-phenyl)-2-ureido-thiophene-3-carb oxylic acid (S)-piperidin-3-ylamide;
1.7: 5-[4-(Piperidine-1-carbonyl)-phenyl]-2-ureido-thiophene-3-carb oxylic acid (S)-piperidin-3-ylamide;
1.8: 5-(4-Cyano-phenyl)-3-ureido-thiophene-2-carboxylic acid (S)-piperidin-3-ylamide;
1.9: 5-[4-(Piperidine-1-carbonyl)-phenyl]-3-ureido-thiophene-2-carb oxylic acid (S)-piperidin-3-ylamide;
1.10: 5-(3,4-Difluoro-phenyl)-3-ureido-thiophene-2-carboxylic acid (S)-piperidin-3-ylamide;
1.11: 5-(3-Chloro-phenyl)-3-ureido-thiophene-2-carboxylic acid (S)-piperidin-3-ylamide;
1.12: 5-(2,3-Difluoro-phenyl)-3-ureido-thiophene-2-carboxylic acid (S)-piperidin-3-ylamide;
1.13: 5-(2,4-Difluoro-phenyl)-3-ureido-thiophene-2-carboxylic acid (S)-piperidin-3-ylamide;
1.14: 5-(3,5-Difluoro-phenyl)-3-ureido-thiophene-2-carboxylic acid (S)-piperidin-3-ylamide;
1.15: 5-Phenyl-3-ureido-thiophene-2-carboxylic acid (S)-piperidin-3-ylamide;
1.16: 5-(4-Chloro-phenyl)-3-ureido-thiophene-2-carboxylic acid (S)-piperidin-3-ylamide.

Additional CHK1 inhibitors include the substituted heterocycles of Formula (II) disclosed in WO2006/106326:

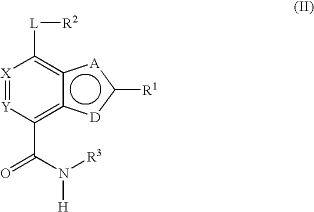

wherein:

A and D are each independently selected from N, CH, S, O and NR⁴;

L is selected from NR⁵, O and S;

X and Y are each independently selected from N and CH;

R¹ is selected from cyano, halo; C₁₋₆alkyl, —NR¹¹R¹², C₁₋₆alkoxy, C₂₋₆alkenyl, C₂₋₆alkynyl, cycloalkyl, cycloalkenyl, aryl, heterocyclyl, OR⁶; —COcarbocyclyl, —COheterocyclyl, —CO(C₁₋₆alkyl), —CONR²⁸R²⁹, —S(O)ₓ(C₁₋₆alkyl), —S(O)ₓcarbocyclyl, —S(O)ₓheterocyclyl, S(O)_{y}NR²⁸R²⁹, and —(C₁₋₆alkyl)S(O)_{y}NR²⁸R²⁹ wherein x is independently 0 to 2 and y is independently 1 or 2; and wherein R¹ may be optionally substituted on one or more carbon atoms by one or more R⁹; and wherein if heterocyclyl contains an —NH— moiety, the nitrogen of said moiety may be optionally substituted by a group selected from R¹⁰);

R² is selected from (C₁₋₃alkyl)NR⁷R⁸, a 4- to 7-membered heterocyclyl ring containing at least one nitrogen atom, —COcarbocyclyl, —COheterocyclyl, —CO(C₁₋₆alkyl), —CONR²⁸R²⁹, —CO₂(C₁₋₆alkyl), —CO₂carbocyclyl, —CO₂heterocyclyl, —CO₂NR²⁸R²⁹, —S(O)ₓ(C₁₋₆alkyl), —S(O)ₓcycloalkyl, —S(O)ₓcycloalkenyl, —S(O)ₓheterocyclyl, S(O)_{y}NR²⁸R²⁹, and —(C₁₋₆alkyl)S(O)_{y}NR²⁸R²⁹ wherein x is independently 0 to 2 and y is independently 1 or 2 and wherein R² may be optionally substituted on one or more carbon atoms by one or more R¹³; and further wherein if heterocyclyl contains an —NH— moiety, the nitrogen of said moiety may be optionally substituted by a group selected from R¹⁴;

R³ is selected from H, benzyl, C₁₋₆alkyl, cycloalkyl, cycloalkenyl, aryl, heterocyclyl, OR⁶, CHO, —COcarbocyclyl, —CO(C₁₋₆alkyl), —CONR²⁸R²⁹, —S(O)ₓ(C₁₋₆alkyl), —S(O)ₓcarbocyclyl, —S(O)ₓheterocyclyl, S(O)_{y}NR²⁸R²⁹, and —(C₁₋₆alkyl)S(O)_{y}NR²⁸R²⁹ wherein x is independently 0 to 2, y is independently 1 or 2 and wherein R³ may be optionally substituted on one or more carbon atoms by one or more R¹⁵; and wherein if heterocyclyl contains a —NH— moiety, the nitrogen may be optionally substituted by a group selected from R¹⁶;

R⁴ is selected from H, C₁₋₃alkyl, cyclopropyl and CF₃;

R⁵ is selected from H, C₁₋₆alkyl, cycloalkyl, cycloalkenyl, heterocyclyl and OR⁶; wherein R⁵ may be optionally substituted on carbon by one or more R¹⁷, and wherein if said heterocyclyl contains a —NH— moiety, the nitrogen of said moiety may be optionally substituted by a group selected from R¹⁸;

R⁶ is selected from H, C₁₋₆alkyl, cycloalkyl, cycloalkenyl, aryl, and heterocyclyl; wherein R⁶ may be optionally substituted on carbon by one or more R¹⁹, and wherein if said heterocyclyl contains a —NH— moiety, the nitrogen of said moiety may be optionally substituted by a group selected from R²⁴;

R⁷ and R⁸ are independently selected from H, C₁₋₆alkyl, cycloalkyl, cycloalkenyl, aryl, and heterocyclyl; wherein R⁷ and R⁸ independently of each other may be optionally substituted on carbon by one or more R²⁰, and wherein if said heterocyclyl contains a —NH— moiety, the nitrogen of said moiety may be optionally substituted by a group selected from R²¹;

R¹¹ and R¹² are independently selected from H, C₁₋₆alkyl, cycloalkyl, cycloalkenyl, aryl, heterocyclyl, wherein R¹¹ and R¹² independently of each other may be optionally substituted on carbon by one or more R³², and wherein if said heterocyclyl contains a —NH— moiety, the nitrogen of said moiety may be optionally substituted by a group selected from R³³;

R⁹, R¹³, R¹⁵, R¹⁷, R¹⁹, R²⁰, R³² and R³⁴ are each independently selected from halo, nitro, —NR²⁸R²⁹, cyano, isocyano, C₁₋₆alkyl, C₂₋₆alkenyl, C₂₋₆alkynyl, aryl, cycloalkyl, cycloalkenyl, heterocyclyl, hydroxy, keto (═O), —O(C₁₋₆alkyl), —Ocarbocyclyl, —Oheterocyclyl, —Oaryl, —OC(O)C₁₋₆alkyl, —NHCHO, —N(C₁₋₆alkyl)CHO, —NHCONR²⁸R²⁹, —N(C₁₋₆alkyl)CONR²⁸R²⁹, —NHCO(C₁₋₆alkyl), —NHCOcarbocyclyl, —NHCO(heterocyclyl), —NHCO₂(C₁₋₆alkyl); —NHCO₂H, —N(C₁₋₆alkyl)CO(C₁₋₆alkyl), —NHSO₂(C₁₋₆alkyl), carboxy, -amidino, —CHO, —CONR²⁸R²⁹, —CO(C₁₋₆alkyl), —COheterocyclyl, —COcycloalkyl, —COcycloalkenyl, —COaryl, —CO₂H, —CO₂(C₁₋₆alkyl), —CO₂carbocyclyl, —CO₂heterocyclyl, —OC(O)(NR²⁸R²⁹), mercapto, —S(O)ₓ(C₁₋₆alkyl), —S(O)ₓcarbocyclyl, —S(O)ₓheterocyclyl, and —S(O)ₓNR²⁸R²⁹; wherein x is independently 0 to 2, wherein R⁹, R¹³, R¹⁵, R¹⁷, R¹⁹, R²⁰, R³² and R³⁴ independently of each other may be optionally substituted on carbon by one or more R²² and wherein if heterocyclyl contains a —NH— moiety, the nitrogen of said moiety may be optionally substituted by a group selected from R²³;

R¹⁰, R⁴⁴, R⁴⁶, R⁴⁸, R²¹, R²⁴, R³³, and R³⁵ are each independently selected from cyano, C₁₋₆alkyl, C₂₋₆alkenyl, C₂₋₆alkynyl, aryl, cycloalkyl, cycloalkenyl, heterocyclyl, hydroxy, —O(C₁₋₆alkyl), —Ocarbocyclyl, -amidino, —CHO, —CONR²⁸R²⁹, —CO(C₁₋₆alkyl), —COheterocyclyl, —COcarbocyclyl —COaryl, —CO₂(C₁₋₆alkyl), —CO₂carbocyclyl, —CO₂heterocyclyl, —S(O)ₓ(C₁₋₆alkyl), —S(O)ₓcarbocyclyl, —S(O)ₓheterocyclyl, and —S(O)_{y}NR²⁸R²⁹; wherein x is independently 0 to 2, and y is independently 1 or 2; wherein R¹⁰, R¹⁴, R¹⁶, R¹⁸, R²¹, R²⁴, R³³ and R³⁵ independently of each other may be optionally substituted on carbon by one or more R²⁵ and wherein if said heterocyclyl contains a —NH— moiety, the nitrogen of said moiety may be optionally substituted by a group selected from R²⁶;

R²² and R²⁵ are each independently selected from halo, nitro, —NR²⁸R²⁹, cyano, isocyano, C₁₋₆alkyl, C₂₋₆alkenyl, C₂₋₆alkynyl, aryl, cycloalkyl, cycloalkenyl, heterocyclyl, hydroxy, keto(═O), —O(C₁₋₆alkyl), —Ocarbocyclyl, —Oheterocyclyl, —Oaryl, —OC(O)C₁₋₆alkyl, —NHCHO, —N(C₁₋₆alkyl)CHO, —NHCONR²⁸R²⁹, —N(C₁₋₆alkyl)CONR²⁸R²⁹, —NHCO(C₁₋₆alkyl), —NHCOcarbocyclyl, —NHCO(heterocyclyl), —NHCO₂(C₁₋₆alkyl); —NHCO₂H, —N(C₁₋₆alkyl)CO(C₁₋₆alkyl), —NHSO₂(C₁₋₆alkyl), carboxy, -amidino, —CHO, —CONR²⁸R²⁹, —CO(C₁₋₆alkyl), —COheterocyclyl, —COcycloalkyl, —COcycloalkenyl, —CO₂H, —CO₂ (C₁₋₆alkyl), —CO₂carbocyclyl, —OC(O)(NR²⁸R²⁹), mercapto, —S(O)ₓ(C₁₋₆alkyl), —S(O)ₓcarbocyclyl, —S(O)ₓheterocyclyl, and —S(O)ₓNR²⁸R²⁹; wherein x is independently 0 to 2, wherein R²² and R²⁵ may be optionally substituted on carbon by one or more R³⁶ and wherein if said heterocyclyl contains a —NH— moiety, the nitrogen of said moiety may be optionally substituted by a group selected from R²⁷;

R²³ and R²⁶ are each independently selected from cyano, C₁₋₆alkyl, C₂₋₆alkenyl, C₂₋₆alkynyl, aryl, cycloalkyl, cycloalkenyl, heterocyclyl, hydroxy, —O(C₁₋₆alkyl), —Ocarbocyclyl, -amidino, —CHO, —CONR²⁸R²⁹, —CO(C₁₋₆alkyl), —COheterocyclyl, —COcycloalkyl, —COcycloalkenyl, —CO₂ (C₁₋₆alkyl), —CO₂carbocyclyl, —S(O)ₓ(C₁₋₆alkyl), —S(O)ₓcarbocyclyl, —S(O)ₓheterocyclyl, and —S(O)_{y}NR²⁸R²⁹; wherein x is independently 0 to 2, and y is independently 1 or 2; wherein R²³ and R²⁶ independently of each other may be optionally substituted on carbon by one or more R³⁰ and wherein if said heterocyclyl contains a —NH— moiety, the nitrogen of said moiety may be optionally substituted by a group selected from R³¹;

R²⁸ and R²⁹ are each independently selected from H, amino, cyano, C₁₋₆alkyl, C₂₋₆alkenyl, C₂₋₆alkynyl, aryl, cycloalkyl, cycloalkenyl, heterocyclyl, hydroxy, —O(C₁₋₆alkyl), —Oaryl, —OCOalkyl, -amidino, —CHO, —CO(C₁₋₆alkyl), —COheterocyclyl, —COcycloalkyl, —COcycloalkenyl, —SO(C₁₋₆alkyl), —SO₂ (C₁₋₆alkyl), wherein R²⁸ and R²⁹ independently of each other may be optionally substituted on carbon by one or more R³⁴; and wherein if said heterocyclyl contains a —NH— the nitrogen of said moiety may be optionally substituted by a group selected from R³⁵;

R³⁰ and R³⁶ are each independently selected from halo, nitro, —NR²⁸R²⁹, cyano, isocyano, C₁₋₆alkyl, C₂₋₆alkenyl, C₂₋₆alkynyl, aryl, cycloalkyl, cycloalkenyl, heterocyclyl, hydroxy, keto (═O), —O(C₁₋₆alkyl), —Ocarbocyclyl, —OC(O)C₁₋₆alkyl, —NHCHO, —N(C₁₋₆alkyl)CHO, —NHCONR²⁸R²⁹, —N(C₁₋₆alkyl)CONR²⁸R²⁹, —NHCO(C₁₋₆alkyl), —NHCOcarbocyclyl, —NHCO(heterocyclyl), —NHCO₂ (C₁₋₆alkyl); —NHCO₂H, —N(C₁₋₆alkyl)CO(C₁₋₆alkyl), —NHSO₂(C₁₋₆alkyl), carboxy, -amidino, —CHO, —CONR²⁸R²⁹, —CO(C₁₋₆alkyl), —COheterocyclyl, —COcycloalkyl, —COcycloalkenyl, —CO₂H, —CO₂ (C₁₋₆alkyl), —CO₂carbocyclyl, —OC(O)(NR²⁸R²⁹), mercapto, —S(O)ₓ(C₁₋₆alkyl), —S(O)ₓcarbocyclyl, —S(O)ₓheterocyclyl, and —S(O)ₓNR²⁸R²⁹; wherein x is independently 0 to 2;

R²⁷ and R³¹ are each independently selected from cyano, C₁₋₆alkyl, C₂₋₆alkenyl, C₂₋₆alkynyl, aryl, cycloalkyl, cycloalkenyl, heterocyclyl, hydroxy, —O(C₁₋₆alkyl), —Ocarbocyclyl, —(C₁₋₆alkyl)-O—(C₁₋₆alkyl), -amidino, —CHO, —CONR²⁸R²⁹, —CO(C₁₋₆alkyl), —COheterocyclyl, —COcycloalkyl, —COcycloalkenyl, —CO₂(C₁₋₆alkyl), —CO₂carbocyclyl, —S(O)ₓ(C₁₋₆alkyl), —S(O)ₓcarbocyclyl, —S(O)ₓheterocyclyl, and —S(O)_{y}NR²⁸R²⁹; wherein x is independently 0 to 2, and y is independently 1 or 2;

or a pharmaceutically acceptable salt thereof.

Compounds of Formula (II) which are of particular interest include the following:
1.17: 2-phenyl-4-[(3S)-piperidin-3-ylamino]thieno[3,2-c]pyridine-7-carboxamide;
1.18: 4-[(3S)-piperidin-3-ylamino]-2-(3-thienyl)thieno[3,2-c]pyridine-7-carboxamide;
1.19: 2-(3-fluorophenyl)-4-[(3S)-piperidin-3-ylamino]thieno[3,2-c]pyridine-7-carboxamide;
1.20: 4-[(3S)-piperidin-3-ylamino]-2-(2-thienyl)thieno[3,2-c]pyridine-7-carboxamide;
1.21: 2-(4-fluorophenyl)-4-[(3S)-piperidin-3-ylamino]thieno[3,2-c]pyridine-7-carboxamide;
1.22: 2-(3,4-difluorophenyl)-4-[(3S)-piperidin-3-ylamino]thieno[3,2-c]pyridine-7-carboxamide;
1.23: 2-(1-benzyl-1H-pyrazol-4-yl)-4-[(3S)-piperidin-3-ylamino]thieno[3,2-c]pyridine-7-carboxamide;
1.24: 4-{methyl[(3S)-piperidin-3-yl]amino}-2-phenylthieno[3,2-c]pyridine-7-carboxamide;
1.25: 2-(3-fluorophenyl)-4-{methyl[(3S)-piperidin-3-yl]amino}thieno[3,2-c]pyridine-7-carboxamide;
1.26: 2-(4-fluorophenyl)-4-{methyl[(3S)-piperidin-3-yl]amino}thieno[3,2-c]pyridine-7-carboxamide;
1.27: 4-{methyl[(3S)-piperidin-3-yl]amino}-2-(3-thienyl)thieno[3,2-c]pyridine-7-carboxamide;
1.28: 4-{[trans-2-methylpiperidin-3-yl]amino}-2-phenylthieno[3,2-c]pyridine-7-carboxamide;
1.29: 2-(3-fluorophenyl)-4-{[trans-2-methylpiperidin-3-yl]amino}thieno[3,2-c]pyridine-7-carboxamide;
1.30: 4-{[trans-2-methylpiperidin-3-yl]amino}-2-(3-thienyl)thieno[3,2-c]pyridine-7-carboxamide;
1.31: 2-(4-fluorophenyl)-4-{[trans-2-methylpiperidin-3-yl]amino}thieno[3,2-c]pyridine-7-carboxamide;
1.32: 4-{[(2R,3S)-2-methylpiperidin-3-yl]amino}-2-phenylthieno[3,2-c]pyridine-7-carboxamide;
1.33: 4-{[(2R,3S)-2-methylpiperidin-3-yl]amino}-2-(3-thienyl)thieno[3,2-c]pyridine-7-carboxamide;
1.34: 4-{methyl[trans-2-methylpiperidin-3-yl]amino}-2-phenylthieno[3,2-c]pyridine-7-carboxamide;
1.35: 4-[(2,6-dimethylpiperidin-3-yl)amino]-2-phenylthieno[3,2-c]pyridine-7-carboxamide;
1.36: 4-[(2,6-dimethylpiperidin-3-yl)amino]-2-(3-fluorophenyl)thieno[3,2-c]pyridine-7-carboxamide;
1.37: 4-[(2,6-dimethylpiperidin-3-yl)amino]-2-(3-thienyl)thieno[3,2-c]pyridine-7-carboxamide;
1.38: 4-[(6-methylpiperidin-3-yl)amino]-2-(3-thienyl)thieno[3,2-c]pyridine-7-carboxamide;
1.39: 4-[(6-methylpiperidin-3-yl)amino]-2-phenylthieno[3,2-c]pyridine-7-carboxamide;
1.40: 2-{4-[(dimethylamino)methyl]phenyl}-4-[(3S)-piperidin-3-ylamino]thieno[3,2-c]pyridine-7-carboxamide;
1.41: 4-[(3S)-piperidin-3-ylamino]-2-[4-(piperidin-1-ylmethyl)phenyl]thieno[3,2-c]pyridine-7-carboxamide;
1.42: 2-[4-(morpholin-4-ylmethyl)phenyl]-4-[(3S)-piperidin-3-ylamino]thieno[3,2-c]pyridine-7-carboxamide;
1.43: 2-(4-chlorophenyl)-4-(piperidin-3-ylamino)-1H-indole-7-carboxamide;
1.44: 2-(4-fluorophenyl)-4-(piperidin-3-ylamino)-1H-indole-7-carboxamide;
1.45: 2-phenyl-4-[(3S)-piperidin-3-ylamino]-1H-indole-7-carboxamide;
1.46: 2-(3-fluorophenyl)-4-[(3S)-piperidin-3-ylamino]-1H-indole-7-carboxamide;
1.47: 2-(4-chlorophenyl)-4-[(3S)-piperidin-3-ylamino]-1H-indole-7-carboxamide;
1.48: 2-(4-fluorophenyl)-4-[(3S)-piperidin-3-ylamino]-1H-indole-7-carboxamide;
1.49: 4-{ethyl[(3S)-piperidin-3-yl]amino}-2-phenylthieno[3,2-c]pyridine-7-carboxamide;
1.50: 4-{ethyl[(3S)-piperidin-3-yl]amino}-2-(3-thienyl)thieno[3,2-c]pyridine-7-carboxamide;
1.51: 4-[(trans-2-ethylpiperidin-3-yl)amino]-2-phenylthieno[3,2-c]pyridine-7-carboxamide;
1.52: 4-[(cis-2-ethylpiperidin-3-yl)amino]-2-phenylthieno[3,2-c]pyridine-7-carboxamide.

Compounds of Formulae 1.17-1.52 may be prepared in a number of ways well known to one skilled in the art of organic synthesis, including, but not limited to, as described in WO2006/106326 and as provided herein below.

Formula 1.17, 2-phenyl-4-[(3S)-piperidin-3-ylamino]thieno[3,2-c]pyridine-7-carboxamide, may be prepared as described below:

### Step 1:

(2Z)-3-cyano-3-(2-thienyl)acrylic acid. To a stirred solution of 2-thienylacetonitrile (24.8 g, 0.20 mol) in MeOH (300 mL) is added glyoxylic acid monohydrate (18.5 g, 0.20 mol) and potassium carbonate (25.5 g, 0.20 mol). The reaction slurry is placed under a nitrogen atmosphere and heated to reflux. After 2 h the reaction mixture is cooled to rt and the product is obtained by filtration. The filter cake is washed with a large amount of MeOH and then dried in a vacuum oven overnight to give 43.1 g (99%) of the title compound as a white crystalline potassium salt.

### Step 2:

(2Z)-3-cyano-3-(2-thienyl)acryloyl chloride. To a stirred solution of oxalyl chloride (2.6 mL, 30 mmol) in 10 mL of CH₂Cl₂ is added a solution of (2Z)-3-cyano-3-(2-thienyl)acrylic acid potassium salt (2.2 g, 12.3 mmol) dissolved in 20 mL of CH₂Cl₂. An additional amount of CH₂Cl₂ is added until the viscous heterogeneous reaction mixture can be stirred easily. The reaction is stirred for about 1 h at rt. The solids are removed by filtration and washed with generous amounts of CH₂Cl₂. The filtrate and washes are combined and concentrated in vacuo to yield 2.0 g of the title compound that is used in the next step.

### Step 3:

(2Z)-3-cyano-3-(2-thienyl)acryloyl azide. To a rapidly stirred suspension of sodium azide (2.0 g, 30 mmol) in a 50:50 mixture of dioxane/water (20 mL) at 0° C. is added a solution of (2Z)-3-cyano-3-(2-thienyl)acryloyl chloride (2.0 g, 12.3 mmol) dissolved in 10 mL of dioxane. The reaction is stirred for 30 min. at 0° C. and then allowed to reach rt after 1-2 h further stirring. The reaction is then added to ˜100 mL of water. The precipitate formed is filtered, washed with water, and dried in the vacuum oven overnight to give the title azide as a white solid (2.0 g), which is used in the next step without further purification.

### Step 4:

4-oxo-4,5-dihydrothieno[3,2-c]pyridine-7-carbonitrile. A mixture of diphenyl ether (260 mL) and Bu₃N (53 mL) is heated to 210° C. under a stream of nitrogen. A slurry of (2Z)-3-cyano-3-(2-thienyl)acryloyl azide (15.0 g, 73.5 mmol) in CH₂Cl₂ (30 mL) is added drop wise over 2 h (vigorous evolution of N₂ gas). After the addition is complete the reaction is stirred at 210° C. for a further 10 min, then the reaction is allowed to cool to rt, then in an ice bath. Hexanes (500 mL) is added and the precipitate is filtered off under suction, washing with copious quantities of hexanes. The obtained solid is dried in a vacuum oven overnight (without heating) to obtain the title compound as a pale brown solid (9.89 g, 76%).

### Step 5:

2-bromo-4-oxo-4,5-dihydrothieno[3,2-c]pyridine-7-carbonitrile. A solution of 4-oxo-4,5-dihydrothieno[3,2-c]pyridine-7-carbonitrile (0.8 g, 4.5 mmol) in a 50:50 mixture of DMF/Acetic Acid (20 mL) is charged with N-bromosuccinimide (1.6 g, 9 mmol). The dark reaction mixture is heated to 80° C. for 12 h. After cooling to rt, the reaction is added to ˜100 mL of water while stirring. The pH of the cloudy solution is adjusted to 9-10 with sat. NaHCO₃. The product is obtained by filtration, washed with water, and is dried in a vacuum oven (1.1 g, 100%).

### Step 6:

2-bromo-4-chlorothieno[3,2-c]pyridine-7-carbonitrile. A solution of 2-bromo-4-oxo-4,5-dihydrothieno[3,2-c]pyridine-7-carbonitrile (1.1 g, 4.5 mmol) dissolved in POCl₃ (10 mL) is heated to reflux overnight. After cooling to rt, the reaction is concentrated to dryness under vacuum. The solids are slowly and carefully suspended in ˜50-100 mL of water. The product is obtained by filtration, followed by washing with water, saturated NaHCO₃, water, and drying in a vacuum oven (1.0 g, 83%).

### Step 7:

tert-butyl (3S)-3-[(7-cyano-2-bromothieno[3,2-c]pyridin-4-yl)amino]piperidine-1-carboxylate. To a stirred solution of 2-bromo-4-chlorothieno[3,2-c]pyridine-7-carbonitrile (0.48 g, 1.76 mmol) and tert-butyl (3S)-3-aminopiperidine-1-carboxylate (0.40 g, 2.0 mmol) in NMP (5 mL) is added potassium carbonate (0.5 g, 3.52 mmol). The heterogeneous mixture is heated to 80° C. for 2 h, cooled to rt, and then added to ˜50 mL of water. The product (880 mg) is isolated by filtration and dried. The title compound is further purified using MPLC (SiO₂; 30-50% EtOAc/Hexanes gradient) to give 0.54 g, 70% as a light yellow crystalline solid.

### Step 8:

tert-butyl (3S)-3-{[7-cyano-2-(phenyl)thieno[3,2-c]pyridin-4-yl]amino}piperidine-1-carboxylate. A mixture of tert-butyl (3S)-3-[(7-cyano-2-bromothieno[3,2-c]pyridin-4-yl)amino]piperidine-1-carboxylate (0.18 g. 0.41 mmol), phenylboronic acid (0.076 g, 0.62 mmol), palladium(0)tetrakistriphenylphosphine (Pd(PPh₃)₄), (0.10 g, 0.062 mmol), and cesium carbonate (0.41 g, 1.25 mmol), are dissolved in water (1 mL), and dioxane (3 mL). This reaction mixture is stirred at 80° C. for 1 h under a nitrogen atmosphere, and then allowed to cool to rt. The water is removed with a pipette and dioxane is removed under vacuum. The residue is purified by MPLC (SiO₂; 30-60% EtOAc/Hexanes) gave the title compound (140 mg, 78%).

### Step 9:

2-phenyl-4-[(3S)-piperidin-3-ylamino]thieno[3,2-c]pyridine-7-carboxamide. A solution of tert-butyl (3S)-3-{[7-cyano-2-(phenyl)thieno[3,2-c]pyridin-4-yl]amino}piperidine-1-carboxylate (80 mg, 0.18 mmol) and 12N HCl (conc., 4 mL) is stirred for 24 hours. Water (10-20 mL) is added and the pH of the solution is adjusted to 10-11 with sat. NaHCO₃. The material is isolated by filtration and is washed with a small amount of cold water. The material is dried and then purified by MPLC (SiO₂; NH₄OH/MeOH/CH₂Cl₂; 2:10:88) to give the title compound (24 mg, 38%).

Formulae 1.18-1.23 may be made in a similar fashion as Formula 1.17 using appropriate starting materials familiar to one of skill in the art.

The compound of Formula 1.24, 4-{methyl[(3S)-piperidin-3-yl]amino}-2-phenylthieno[3,2-c]pyridine-7-carboxamide, may be prepared in a similar fashion to Formula 1.17 but using tert-butyl (3S)-3-(methylamino)piperidine-1-carboxylate (synthesis described below) as the starting material in step 7:

tert-butyl (3S)-3-(methylamino)piperidine-1-carboxylate. To a solution of formaldehyde (37%, aq.; 0.37 ml, 4.7 mmol) in 20 ml dry MeOH containing 3 Å molecular sieves is added tert-butyl (3S)-3-aminopiperidine-1-carboxylate (1.0 g, 5 mmol). The reaction is stirred under N₂ at rt for ˜30 h, and then NaBH₄ (304 mg, 8 mmol) is added as a solid. The reaction is stirred at rt overnight and then quenched with 1N NaOH (˜10 ml). The phases are separated and the remaining aqueous layer is extracted with ether (3×). The combined organic layers are washed with water and brine, dried, and evaporated to yield a colorless oil (1.04 g, 100%).

Formulae 1.25-1.27 may be made in a similar fashion as Formula 1.24 using appropriate starting materials familiar to one of skill in the art.

Formula 1.28, 4-{[trans-2-methylpiperidin-3-yl]amino}-2-phenylthieno[3,2-c]pyridine-7-carboxamide, may be prepared in a similar fashion to Formula 1.17 but using benzyl trans-3-amino-2-methylpiperidine-1-carboxylate (synthesis described below) as the starting material in step 7:

tert-butyl ((1S)-1-acetyl-4-{[(benzyloxy)carbonyl]amino}butyl)carbamate. To a 3-necked flask containing N⁵-[(benzyloxy)carbonyl]-N²-(tert-butoxycarbonyl)-L-ornithine (36.6 g, 100 mmol) equipped with a magnetic stir bar and an addition funnel is added dry THF (100 mL). The addition funnel is charged with MeLi (1.6M in ether; 275 mL; 440 mmol), which is subsequently added slowly (over 20 minutes) to the reaction mixture cooled to 0° C. This solution is then warmed to rt. After stirring for an additional 5 h, the reaction is quenched by pouring onto a stirred ice/water mixture. The aqueous mixture is extracted with EtOAc (3×100 mL). The combined organic layers are then washed with brine, dried over Na₂SO₄, filtered and concentrated in vacuo to yield a yellow oil (6.0 g, 98%). After purification using MPLC (SiO₂; 25-60% EtOAc/Hexanes), the product is isolated as a clear oil (5.2 g, 14%).

tert-butyl [trans-2-methylpiperidin-3-yl]carbamate. To a stirred solution of tert-butyl ((1S)-1-acetyl-4-{[(benzyloxy)carbonyl]amino}butyl)carbamate (3.9 g, 10.7 mmol) in MeOH (200 mL) is added 10% Pd/C (0.1 mmol). The heterogeneous mixture is hydrogenated at atmospheric pressure for 3 days (or 40 psi overnight). The product is isolated as clear oil after filtration through diatomaceous earth and evaporation of the filtrate to give the title compound (2.3 g; 100%), which is used in the next step without purification.

benzyl trans-3-[(tert-butoxycarbonyl)amino]-2-methylpiperidine-1-carboxylate. To a stirred solution of tert-butyl [trans-2-methylpiperidin-3-yl]carbamate (2.3 g, 10.7 mmol) and diisopropylethylamine (2.1 mL, 12 mmol) dissolved in CH₂Cl₂ (40 mL) cooled to 0° C. is added benzyl chloroformate (1.7 mL, 12 mmol). The reaction mixture is then warmed to rt and stirred for an additional 1 h. The mixture is then diluted with CH₂Cl₂ and washed with 1N HCl and brine, dried over Na₂SO₄, filtered and concentrated in vacuo to yield a yellow oil. After purification using MPLC (SiO₂; 10-40% EtOAc/Hexanes), the title compound (trans diastereomer) is isolated as a crystalline solid (1.8 g). The cis diastereomer, benzyl cis-3-[(tert-butoxycarbonyl)amino]-2-methylpiperidine-1-carboxylate is also isolated pure (1.3 g).

benzyl trans-3-amino-2-methylpiperidine-1-carboxylate. To a solution of benzyl trans-3-[(tert-butoxycarbonyl)amino]-2-methylpiperidine-1-carboxylate (1.8 g, 5.2 mmol) dissolved in MeOH (10 mL) is added HCl (4N in dioxane; 20 mL). After stirring for 1 h at rt, the reaction is concentrated in vacuo, redissolved in MeOH, and then concentrated in vacuo to yield the hydrochloride salt of the title compound as a clear crystalline solid (1.46 g, 100%).

Compounds of Formulae 1.29-1.31 may be prepared in a similar fashion to Formula 1.17 but using benzyl cis-3-amino-2-methylpiperidine-1-carboxylate (described below) as the starting material in step 7:

benzyl cis-3-amino-2-methylpiperidine-1-carboxylate. To a solution of benzyl cis-3-[(tert-butoxycarbonyl)amino]-2-methylpiperidine-1-carboxylate (1.2 g, 3.4 mmol) dissolved in MeOH (10 mL) is added HCl (4N in dioxane; 20 mL). After stirring for 1 h at rt, the reaction is concentrated in vacuo, redissolved in MeOH, and then concentrated in vacuo to yield the hydrochloride salt of the title compound as a clear crystalline solid (0.97 g, 100%).

Formula 1.32 may be prepared by chiral preparatory HPLC separation of the compound of Formula 1.28.

Formula 1.33 may be prepared by chiral preparatory HPLC separation of the compound of Formula 1.30.

Formula 1.34, 4-{methyl[trans-2-methylpiperidin-3-yl]amino}-2-phenylthieno[3,2-c]pyridine-7-carboxamide, may be prepared in a similar fashion to Formula 1.17 but using benzyl trans-2-methyl-3-(methylamino)piperidine-1-carboxylate (synthesis described below) as the starting material in step 7:

benzyl trans-3-[(tert-butoxycarbonyl)(methyl)amino]-2-methylpiperidine-1-carboxylate. To a solution of benzyl trans-3-[(tert-butoxycarbonyl)amino]-2-methylpiperidine-1-carboxylate (0.10 g, 0.29 mmol) in 10 mL dry THF under a N₂ atmosphere is added sodium hydride (60% in mineral oil; 8 mg, 0.32 mmol). This solution is stirred for 30 minutes and then methyl iodide (0.017 mL, 0.287 mmol) is added. The reaction mixture is stirred for two hours and 10 mL of MeOH is added slowly to quench the reaction. The contents are CIV and the residue is dissolved in 30 mL CH₂Cl₂ and washed with water (2×). The organic layer is CIV to yield 0.16 g of the title product.

benzyl trans-2-methyl-3-(methylamino)piperidine-1-carboxylate. To a solution of benzyl trans-3-[(tert-butoxycarbonyl)(methyl)amino]-2-methylpiperidine-1-carboxylate (0.16 g 0.45 mmol) dissolved in MeOH (4 mL) is added HCl (4N in dioxane; 4 mL). After stirring for 2 h at rt, the reaction is concentrated in vacuo, redissolved in MeOH, and then concentrated in vacuo to yield the hydrochloride salt of the title compound as an oily crystalline solid (0.12 g).

Formula 1.35, 4-[(2,6-dimethylpiperidin-3-yl)amino]-2-phenylthieno[3,2-c]pyridine-7-carboxamide, may be prepared as follows:

2,6-dimethylpiperidin-3-amine. To a high-pressure vessel containing 2,6-dimethylpyridin-3-amine (2.08 g, 17.0 mmol) is added water and 12 N HCl (10 mL each) followed by platinum (IV) oxide (500 mg, 2.20 mmol) under nitrogen. The high-pressure vessel is then evacuated under reduced pressure and placed on a Parr hydrogenation apparatus at 50 psi for 48 hours. The mixture is evacuated under nitrogen, filtered over a bed of diatomaceous earth, and rinsed with copious amounts of MeOH. The collected filtrate is concentrated in vacuo to afford the title compound that is used directly in the next reaction as a mixture of isomers.

2-bromo-4-[(2,6-dimethylpiperidin-3-yl)amino]thieno[3,2-c]pyridine-7-carbonitrile. To 2,6-dimethylpiperidin-3-amine (1.23 g, 4.50 mmol) dissolved in NMP (10 mL) is added potassium carbonate (1.87 g, 13.5 mmol) and 2-bromo-4-chlorothieno[3,2-c]pyridine-7-carbonitrile (1.23 g, 4.50 mmol). The resulting mixture is heated to 80° C. and stirred for twelve hours or until LCMS indicates complete conversion to product. The mixture is then diluted with water (100 mL) and the resulting solid is filtered, washed with water (20 mL) and dried under reduced pressure for up to eight hours.

4-[(2,6-dimethylpiperidin-3-yl)amino]-2-phenylthieno[3,2-c]pyridine-7-carbonitrile. To a solution containing 2-bromo-4-[(2,6-dimethylpiperidin-3-yl)amino]thieno[3,2-c]pyridine-7-carbonitrile (400 mg, 1.09 mmol), phenylboronic acid (200 mg, 1.64 mmol), cesium carbonate (1.06 g, 3.27 mmol), and dioxane/water (2 mL/1 mL) is added Pd(PPh₃)₄ (126 mg, 0.109 mmol). The reaction is heated to 80° C. for one hour whereupon the reaction is cooled to rt, filtered, and purified using silica gel chromatography (100% CH₂Cl₂ to 20% MeOH/CH₂Cl₂/3% NH₄OH) to afford the title compound.

4-[(2,6-dimethylpiperidin-3-yl)amino]-2-phenylthieno[3,2-c]pyridine-7-carboxamide. To a flask containing 4-[(2,6-dimethylpiperidin-3-yl)amino]-2-phenylthieno[3,2-c]pyridine-7-carbonitrile is added 5.00 mL of 12 N HCl. The reaction mixture is stirred at rt and monitored by LCMS. Additional 12 N HCl is added every twelve hours to afford complete conversion to the desired product. Upon completion, the reaction mixture is diluted with MeOH and concentrated under reduced pressure to yield the product, which is purified by preparatory HPLC (5%-95% H₂O/MeCN/0.1% TFA) to afford the title compound as a mixture of isomers.

The compounds of Formulae 1.36-1.39 may be prepared in a similar fashion to that of Formula 1.35 using appropriate starting materials familiar to one of skill in the art.

The compound 2-{3-[(dimethylamino)methyl]phenyl}-4-[(3S)-piperidin-3-ylamino]thieno[3,2-c]pyridine-7-carboxamide may be prepared as follows:

tert-butyl (3S)-3-{[7-cyano-2-(3-formylphenyl)-1-benzothien-4-yl]amino}piperidine-1-carboxylate. A mixture of tert-butyl (3S)-3-[(7-cyano-2-bromothieno[3,2-c]pyridin-4-yl)amino]piperidine-1-carboxylate (500 mg, 1.1 mmol), 3-formylphenylboronic acid (257 mg, 1.7 mmol), cesium carbonate (1.12 g, 3.4 mmol) and Pd(PPh₃)₄ (198 mg, 0.17 mmol) are heated to 80° C. in dioxane (8.2 mL) and H₂O (2.7 mL). After 30 min, the solution is cooled to rt, the water layer is removed via pipette and the solution is concentrated in vacuo. The residue is purified by MPLC (SiO₂; 20-50% EtOAc/hexanes) to yield the title compound (185 mg, 35%).

tert-butyl (3S)-3-[(7-cyano-2-{4-[(dimethylamino)methyl]phenyl}-1-benzothien-4-yl)amino]piperidine-1-carboxylate. A solution of tert-butyl (3S)-3-{[7-cyano-2-(3-formylphenyl)-1-benzothien-4-yl]amino}piperidine-1-carboxylate (50 mg, 0.11 mmol) and dimethylamine (0.54 mL of a 2 M solution in THF, 1.1 mmol) are stirred in ethylene glycol dimethyl ether (0.54 mL) at rt. Acetic Acid (2 drops) is added, followed by NaBH(OAc)₃ (92 mg, 0.43 mmol). The solution is heated to 80° C. for 30 min. The solution is then cooled to rt. Water (2 drops) is added, followed by 1 M NaOH (1 mL). The product is extracted with EtOAc. The organic extracts are washed with brine, dried over MgSO₄ and concentrated in vacuo to yield the title compound, which is used directly in the next step.

2-{3-[(dimethylamino)methyl]phenyl}-4-[(3S)-piperidin-3-ylamino]thieno[3,2-c]pyridine-7-carboxamide. tert-Butyl (3S)-3-[(7-cyano-2-{4-[(dimethylamino)methyl]phenyl}-1-benzothien-4-yl)amino]piperidine-1-carboxylate is dissolved in 12 M HCl (4 mL) and kept at rt for 17 h. The solution is concentrated in vacuo, and azeotroped with MeOH to yield the title compound as the hydrochloride salt (48 mg, 98%).

The compounds of Formulae 1.40-1.42 may be prepared in a similar fashion to 2-{3-[(dimethylamino)methyl]phenyl}-4-[(3S)-piperidin-3-ylamino]thieno[3,2-c]pyridine-7-carboxamide from appropriate materials which would be familiar to one of skill in the art.

The compound 2-(3-fluorophenyl)-7-[(3S)-piperidin-3-ylamino]thieno[2,3-c]pyridine-4-carboxamide may be prepared as follows:

(2Z)-3-cyano-3-(3-thienyl)acrylic acid. To 3-thiopheneacetonitrile (166 mmol) is added glyoxylic acid (174 mmol), MeOH (332 mL) and potassium carbonate (174 mmol). The resulting mixture is heated to reflux for three hours followed by cooling to rt. The resultant solid is filtered, rinsed with MeOH, and dried in a vacuum oven to afford the title compound (26.6 g, 90% yield).

(2Z)-3-cyano-3-(3-thienyl)acryloyl chloride. To a solution of oxalyl chloride (27.3 mL, 313 mmol) in CH₂Cl₂ (57 mL) is added (2Z)-3-cyano-3-(3-thienyl)acrylic acid (26.6 g, 149 mmol) in portions. The resulting solution is stirred at rt until LCMS indicated completion of the reaction. The reaction mixture is then filtered and rinsed with CH₂Cl₂. The filtrate is collected, concentrated under reduced pressure and dried under vacuum to afford the title compound as a yellow solid which is used directly in the next reaction (18.5 g, 63% yield).

(2Z)-3-cyano-3-(3-thienyl)acryloyl azide. To a solution of sodium azide (12.2 g, 187 mmol) in a 1:1 mixture of dioxane/water (23 mL) is added at 0° C. (2Z)-3-cyano-3-(3-thienyl)acryloyl chloride (18.5 g, 93.5 mmol) in 33 mL dioxane. The reaction is stirred for 15 minutes at 0° C., followed by warming the reaction to rt. After approximately 1.5 hours, water (100 mL) is added to the reaction and the resulting solid is filtered and dried in a vacuum oven to yield the title compound (15.1 g, 82% yield).

7-oxo-6,7-dihydrothieno[2,3-c]pyridine-4-carbonitrile. To a solution of phenyl ether (224 mL) and tributylamine (53.0 mL) at 230° C. is added drop wise (2Z)-3-cyano-3-(3-thienyl)acryloyl azide in approximately 10 mL of CH₂Cl₂. The mixture is stirred at 230° C. for thirty minutes, cooled to rt, followed by the addition of 500 mL hexane, which affords a yellowish solid. The resultant solid is washed with hexane and dried under vacuum to yield the title compound (4.61 g, 44% yield).

2-bromo-7-oxo-6,7-dihydrothieno[2,3-c]pyridine-4-carbonitrile. To a solution of 7-oxo-6,7-dihydrothieno[2,3-c]pyridine-4-carbonitrile (2.30 g, 13.1 mmol) in 1/1 acetic acid/DMF (10 mL) is added N-bromosuccinimide (11.6 g, 65.3 mmol). The reaction mixture is heated to 80° C. for one. The solution is cooled to rt and diluted with 100 mL of water. The reaction is then neutralized with saturated sodium bicarbonate followed by filtration of the resulting solid, which is dried in a vacuum oven to afford the title compound (3.20 g, 96% yield).

2-bromo-7-chlorothieno[2,3-c]pyridine-4-carbonitrile. To 2-bromo-7-oxo-6,7-dihydrothieno[2,3-c]pyridine-4-carbonitrile (3.20 g, 12.5 mmol) is added 45.0 mL of phosphorous oxychloride. The reaction is heated to reflux overnight after which LCMS indicated reaction is complete. The reaction is then cooled to rt and the volatiles are removed under reduced pressure. To the resulting residue is added approximately 200 mL of water. The black solid is filtered and rinsed with copious amounts of water and dried under vacuum to yield the title compound (2.80 g, 82% yield).

tert-butyl (3S)-3-[(2-bromo-4-cyanothieno[2,3-c]pyridin-7-yl)amino]piperidine-1-carboxylate. To a solution of 2-bromo-7-chlorothieno[2,3-c]pyridine-4-carbonitrile (2.80 g, 10.2 mmol) in NMP (10.0 mL) is added potassium carbonate (4.23 g, 30.6 mmol) and tert-butyl (3S)-3-aminopiperidine-1-carboxylate (4.92 g, 24.6 mmol). The reaction mixture is heated to 130° C. until LCMS indicates the reaction is complete. The reaction mixture is then cooled to rt and approximately 100 mL of water is added. The resulting solid is filtered and vacuum dried to afford the title compound.

tert-butyl (3S)-3-{[4-cyano-2-(3-fluorophenyl)thieno pyridin-7-yl]amino}piperidine-1-carboxylate. To tert-butyl (3S)-3-[(2-bromo-4-cyanothieno[2,3-c]pyridin-7-yl)amino]piperidine-1-carboxylate (428 mg, 0.979 mmol) is added cesium carbonate (957 mg, 2.94 mmol), 3-fluorophenyl boronic acid (206 mg, 1.47 mmol), Pd(PPh₃)₄ (113 mg, 0.0979 mmol), and dioxane/water (4 mL/2 mL). The reaction is heated to 80° C. for one hour whereupon the reaction is cooled to rt, filtered, and purified using silica gel chromatography (100% hexanes to 100% EtOAc) to afford the title compound (241 mg, 54% yield).

2-(3-fluorophenyl)-7-[(3S)-piperidin-3-ylamino]thieno pyridine-4-carboxamide. To a flask containing tert-butyl (3S)-3-{[4-cyano-2-(3-fluorophenyl)thieno[2,3-c]pyridin-7-yl]amino}piperidine-1-carboxylate is added approximately 2.00 mL of PPA. The reaction mixture is stirred at 110° C. for 12 hours. The reaction mixture is diluted with 10.0 mL of water and brought to a basic pH with 6N NaOH. The mixture is then extracted with EtOAc (4×100 mL) followed by CH₂Cl₂/MeOH (1/1, 4×100 mL), dried over MgSO₄, and concentrated under reduced pressure to yield the product that is purified by silica gel chromatography (100% CH₂Cl₂ to 20% MeOH/CH₂Cl₂/3% NH₄OH) to afford the title compound.

The compounds of Formulae 1.43 and 1.44 may be prepared in an analogous fashion to 2-(3-fluorophenyl)-7-[(3S)-piperidin-3-ylamino]thieno[2,3-c]pyridine-4-carboxamide using the appropriate starting materials familiar to one of skill in the art.

The compound 2-phenyl-4-(piperidin-3-ylamino)-1H-indole-7-carboxamide may be prepared as follows:

methyl 2-amino-4-nitrobenzoate. To a solution of 2-amino-4-nitrobenzoic acid (24 g, 0.132 mol) in MeOH (500 mL) is slowly added thionyl chloride (96 mL). The resulting solution is refluxed overnight. Upon cooling, the crystalline product is isolated by filtration and drying under high vacuum (22.9 g, 88%).

4-nitro-2-phenyl-1H-indole-7-carboxylic acid. To a solution of methyl 2-amino-4-nitrobenzoate (2.2 g, 11.2 mmol) and acetophenone (2.8 g, 23.3 mmol) in DMSO (30 mL) cooled to −15° C. is added solid KOtBu (2.7 g, 24 mmol). After stirring for 20 min., and then another 2 h at rt, the reaction is quenched with sat. NH₄Cl (200 mL) and then stirred for an additional 1 h at rt. The red precipitate is filtered, washed with water, and dried under high vacuum to give the title compound (2.85 g, 90%).

4-nitro-2-phenyl-1H-indole-7-carboxamide. To a solution of 4-nitro-2-phenyl-1H-indole-7-carboxylic acid (0.60 g, 2.1 mmol) and N-methylmorpholine (2.3 mmol) in CH₂Cl₂ (20 mL) at −15° C. is added isobutyl chloroformate (0.5 mL, 3.8 mmol). After stirring for 1 h, NH₃ (g) is bubbled through the reaction mixture for 10-15 min. and then stirred for an additional 1 h at rt. After removing the solvent, the residue is purified by MPLC (SiO₂; 50-100% EtOAc/Hexanes) to give the product as a dark yellow solid (0.50 g, 85%).

4-amino-2-phenyl-1H-indole-7-carboxamide. To a nitrogen-purged stirred solution of 4-nitro-2-phenyl-1H-indole-7-carboxamide (0.50 g, 17.8 mmol) dissolved in MeOH (30 mL) is added 10% Pd/C (30 mg). The resultant heterogeneous mixture is affixed with a H₂ (g) balloon. After stirring overnight at rt, the reaction is filtered (0.45 u, Teflon). The filtrate is concentrated in vacuo to give the title compound as a light yellow solid (0.35 g, 80%).

tert-butyl 3-{[7-(aminocarbonyl)-2-phenyl-1H-indol-4-yl]amino}piperidine-1-carboxylate.

To a solution of 4-amino-2-phenyl-1H-indole-7-carboxamide (0.60 g, 2.4 mmol) and tert-butyl 3-oxopiperidine-1-carboxylate (0.6 g, 2.8 mmol) dissolved in AcOH (15 mL) is added Na₂SO₄. The mixture is stirred at rt for 1 h and then slowly charged with sodium triacetoxyborohydride (1.5 g, 7.2 mmol). The reaction is stirred at rt for 1 h. The mixture is diluted with EtOAc and water, washed with sat. NaHCO₃, 1N HCl, and sat. NaCl. The organic layer is dried over Na₂SO₄, filtered, and CIV. The residue is purified by MPLC (SiO₂; 50-80% EtOAc/Hexanes title product as a tan solid (0.3 g, 30%). LCMS (ES, M+H=435; M−H=433).

2-phenyl-4-(piperidin-3-ylamino)-1H-indole-7-carboxamide. A stirred solution of tert-butyl 3-{[7-(aminocarbonyl)-2-phenyl-1H-indol-4-yl]amino}piperidine-1-carboxylate (0.15 g, 0.35 mmol) in MeOH (10 mL) is charged with 4.0 N HCl in dioxane (10 mL). The reaction is stirred for 2 h at rt and then concentrated in vacuo to give the hydrochloride salt. The residue is diluted with 2.0 N NH₃ in MeOH (10 mL) and CIV. The residue is purified by MPLC (SiO₂; 10% MeOH/CH₂Cl₂/1.5% NH₄OH-20% MeOH/CH₂Cl₂/3% NH₄OH) to give the title compound as an off-white solid (90 mg, 78%).

The compounds of Formula 1.45 may be prepared by chiral preparatory HPLC separation of the compound 2-phenyl-4-(piperidin-3-ylamino)-1H-indole-7-carboxamide.

The compound 2-(3-fluorophenyl)-4-(piperidin-3-ylamino)-1H-indole-7-carboxamide may be prepared in an analogous fashion to compounds of Formulae 1.43 and 1.44 using the appropriate starting materials familiar to one of skill in the art.

The compound of Formula 1.46 may be prepared by chiral preparatory HPLC separation of 2-(3-fluorophenyl)-4-(piperidin-3-ylamino)-1H-indole-7-carboxamide.

The compound of Formula 1.47 may be prepared by chiral preparatory HPLC separation of Formula 1.43.

The compound of Formula 1.48 may be prepared by chiral preparatory HPLC separation of Formula 1.44.

Compounds of Formulae 1.49 and 1.50 may be made in a similar fashion to Formula 1.24 using the appropriate starting materials familiar to one of skill in the art.

Compounds of Formulae 1.51 and 1.52 may be made in a similar fashion to Formula 1.28-1.31 using the appropriate starting materials familiar to one of skill in the art.

In addition to the pharmaceutically acceptable salts of all the compounds described herein, pharmaceutically acceptable solvates of the compounds or the salts, are also included within the scope of the present invention.

It is contemplated herein that the compounds disclosed herein include compounds wherein one or more of the atoms is a radioisotope of the same element. This may include, but are not limited to, the radiolabeled B cell depleting antibodies discussed herein.

As specifically contemplated herein, the instant invention includes the following methods: A method to treat a patient suffering from a hematologic malignancy comprising administering to said patient a therapeutically effective amount of a CHK1 inhibitor simultaneously with or consecutively with (e.g., before or after) a B cell depleting antibody (Method 1);

A method to treat a patient suffering from chronic lymphocytic leukemia (CLL) comprising administering to said patient a therapeutically effective amount of a CHK1 inhibitor simultaneously with or consecutively with (e.g. before or after) an anti-CD20 antibody (Method 2);

A method to treat a patient suffering from chronic lymphocytic leukemia (CLL) comprising administering to said patient a therapeutically effective amount of a CHK1 inhibitor simultaneously with or consecutively with (e.g. before or after) rituximab (Method 3);

A method to treat a patient suffering from a B cell related tumor comprising administering to said patient a therapeutically effective amount of a CHK1 inhibitor simultaneously with or consecutively with (e.g. before or after) a B cell depleting antibody (Method 4);

A method to treat a patient suffering from a B cell related tumor comprising administering to said patient a therapeutically effective amount of a CHK1 inhibitor simultaneously with or consecutively with (e.g. before or after) an anti-CD20 antibody (Method 5);

A method to treat a patient suffering from a B cell related tumor comprising administering to said patient a therapeutically effective amount of a CHK1 inhibitor simultaneously with or consecutively with (e.g. before or after) rituximab (Method 6);

A method to treat a patient suffering from a hematologic malignancy or B cell related tumor that is resistant to cancer treatment comprising administering to said patient a therapeutically effective amount of a CHK1 inhibitor (Method 7);

A method to treat a patient suffering from a hematologic malignancy or B cell related tumor that is resistant to cancer treatment comprising administering to said patient a therapeutically effective amount of a CHK1 inhibitor simultaneously with or consecutively with (e.g. before or after) a B cell depleting antibody (Method 8);

A method to treat a patient suffering from a hematologic malignancy or B cell related tumor that is resistant to treatment with a DNA damaging agent comprising administering to said patient a therapeutically effective amount of a CHK1 inhibitor (Method 9);

A method to treat a patient suffering from a hematologic malignancy or B cell related tumor that is resistant to treatment with a DNA damaging agent comprising administering to said patient a therapeutically effective amount of a CHK1 inhibitor simultaneously with or consecutively with (e.g. before or after) a B cell depleting antibody (Method 10);

A method to treat a patient suffering from a hematologic malignancy or B cell related tumor that is resistant to treatment with fludarabine comprising administering to said patient a therapeutically effective amount of a CHK1 inhibitor (Method 11);

A method to treat a patient suffering from a hematologic malignancy or B cell related tumor that is resistant to treatment with fludarabine comprising administering to said patient a therapeutically effective amount of a CHK1 inhibitor simultaneously with or consecutively with (e.g. before or after) an anti-CD20 antibody (Method 12);

A method to treat a patient suffering from a hematologic malignancy or B cell related tumor that is resistant to treatment with fludarabine comprising administering to said patient a therapeutically effective amount of a CHK1 inhibitor simultaneously with or consecutively with (e.g. before or after) rituximab (Method 13).

Thus, it is contemplated herein that the methods of the present invention include methods comprising administering to a patient suffering from a hematologic malignancy (including CLL), or a B cell related tumor (including those hematologic malignancies or B cell related tumors resistant to cancer treatment, e.g., resistant to a DNA damaging agent, e.g., fludarabine) a therapeutically effective amount of a CHK1 inhibitor, including but not limited to, a compound of Formula 1.1-1.52 as defined herein, in free form or in a pharmaceutically acceptable salt form or in the form of a pharmaceutically acceptable solvate of the compound or salt, simultaneously with or consecutively with (e.g., before or after) a B cell depleting antibody (e.g. an anti-CD 20 antibody, e.g., rituximab).

In addition, in another embodiment the invention encompasses the use of a CHK1 inhibitor, in free form or in a pharmaceutically acceptable salt form or in the form of a pharmaceutically acceptable solvate of the compound or salt, in the manufacture of a medicament for use in combination therapy with a B cell depleting antibody for the treatment of a hematologic malignancy or B cell related tumor. In one embodiment the hematologic malignancy or B cell related tumor is resistant to cancer treatment, e.g. resistant to treatment with a DNA damaging agent. In one embodiment the DNA damaging agent is fludarabine. In one embodiment, the hematologic malignancy is CLL. In another embodiment the B cell depleting antibody is an anti-CD20 antibody. In another embodiment the anti-CD20 antibody is rituximab. In another embodiment the CHK1 inhibitor is selected from the group consisting of Formula 1.1-1.52 as defined herein in free form or in a pharmaceutically acceptable salt form or in the form of a pharmaceutically acceptable solvate of the compound or salt.

In another embodiment the invention encompasses use of a CHK1 inhibitor in free form or in a pharmaceutically acceptable salt form or in the form of a pharmaceutically acceptable solvate of the compound or salt for use in combination therapy with a B cell depleting antibody for the treatment of a hematologic malignancy or B cell related tumor. In one embodiment the hematologic malignancy or B cell related tumor is resistant to cancer treatment, e.g. resistant to treatment with a DNA damaging agent. In another embodiment the DNA damaging agent is fludarabine. In one embodiment, the hematologic malignancy is CLL. In another embodiment the B cell depleting antibody is an anti-CD20 antibody. In another embodiment the anti-CD20 antibody is rituximab. In another embodiment the compound is selected from the group consisting of Formulae 1.1-1.52, in free form or in a pharmaceutically acceptable salt form or in the form of a pharmaceutically acceptable solvate of the compound or salt.

In another embodiment the invention encompasses a method to treat a patient suffering from a hematologic malignancy or B cell related tumor comprising administering to said patient a therapeutically effective amount of a CHK1 inhibitor, in free form or in a pharmaceutically acceptable salt form or in the form of a pharmaceutically acceptable solvate of the compound or salt simultaneously with or consecutively with (e.g., before or after) a B cell depleting antibody. In one embodiment the hematologic malignancy or B cell related tumor is resistant to cancer treatment, e.g. resistant to treatment with a DNA damaging agent. In another embodiment the DNA damaging agent is fludarabine. In one embodiment the hematologic malignancy is CLL. In another embodiment the B cell depleting antibody is an anti-CD20 antibody. In another embodiment the anti-CD20 antibody is rituximab. In another embodiment the compound is selected from the group consisting of Formulae 1.1-1.52, in free form or in a pharmaceutically acceptable salt form or in the form of a pharmaceutically acceptable solvate of the compound or salt.

In addition, as further contemplated herein, the invention includes the following methods:

Method 1 wherein the compound is of Formula 1.1-1.52;

Method 2 wherein the compound is of Formula 1.1-1.52;

Method 3 wherein the compound is of Formula 1.1-1.52;

Method 4 wherein the compound is of Formula 1.1-1.52;

Method 5 wherein the compound is of Formula 1.1-1.52;

Method 6 wherein the compound is of Formula 1.1-1.52;

Method 7 wherein the compound is of Formula 1.1-1.52;

Method 8 wherein the compound is of Formula 1.1-1.52;

Method 9 wherein the compound is of Formula 1.1-1.52;

Method 10 wherein the compound is of Formula 1.1-1.52;

Method 11 wherein the compound is of Formula 1.1-1.52;

Method 12 wherein the compound is of Formula 1.1-1.52.

Pharmaceutical compositions comprising the CHK1 inhibitors, (including those specifically disclosed herein), and a B cell depleting antibody, (e.g., an anti-CD 20 antibody, e.g., rituximab,) in combination or association with a pharmaceutically acceptable carrier or diluent for use in the methods or uses described herein, are also fully contemplated.

It is contemplated that the invention described herein is not limited to the particular methodology, protocols, and reagents described as these may vary. It is also to be understood that the terminology used herein is for the purpose of describing particular embodiments only, and is not intended to limit the scope of the present invention in any way.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices and materials are herein described. All publications mentioned herein are hereby incorporated by reference in their entirety for the purpose of describing and disclosing the materials and methodologies that are reported in the publication which might be used in connection with the invention.

### EXAMPLES

### Example 1

### Demonstration of Reduced CLL Tumor Viability when Exposed to a Checkpoint 1 Kinase Inhibitor

### A. In Vitro Studies:

Chronic B cell leukemia cell lines (JVM2 and Mec1) (American Type Culture Collection (ATCC), Manassas, Va.) were plated in 96 well plates and 24 hours later were treated with increasing concentrations of a CHK1 inhibitor (Formula 1.1). Following 48 hour treatment cell viability was measured using Cell Titer-Glo® luminescent cell viability assay (Promega, Madison, Wis.). Potent inhibitory activity was observed with the calculated GI₅₀ for Mec1 and JVM2 to be 0.079 μM and 0.013 μM, respectively.

### B. Ex Vivo Studies:

Blood samples (10 mls) were collected from CLL patients and placed on ice. Whole blood was then diluted with phosphate buffered saline (PBS) (2-3 fold dilution) and carefully layered over 12 mls Ficoll-paque (GE Healthcare, Westborough, Mass.) in a 50 ml conical tube. Samples were then centrifuged at 3500 rpm for 35 minutes without break at room temperature. Plasma was removed and opaque interphase containing mononuclear cells was collected and placed into a clean conical centrifuge tube. Cells were then washed twice with PBS with 1% fetal bovine serum (FBS). Following final wash, cells were resuspended in 5˜10 mls RPMI 1640 (Invitrogen, Grand Island, N.Y.) plus 10% fetal calf serum (FCS) medium. Cells were then counted by Trypan blue exclusion method and viability and purity assessed.

Cells were then plated into 96 well plates (1×10⁵ cells/well) and incubated overnight in a drug free medium. The next morning cells were treated with fludarabine (1, 3 and 10 μM), gemcitabine (0.1 and 1 μM), CHK1 inhibitor of Formula 1.1 (0.01, 0.1 and 0.5 μM) or the combination of fludarabine and CHK1 inhibitor or gemcitabine and CHK1 inhibitor. In addition, cells were treated with a 8-point titration of single agent to determine IC₅₀s (CHK1 inhibitor from 0.1 nM to 500 nM; fludarabine 0.1 μM to 30 μM and gemcitabine 0.01 μM to 10 μM). Following 48 hour treatment, cell viability was measured using Cell Titer-Glo® luminescent cell viability assay (Promega, Madison, Wis.). Data showed a newly diagnosed patient who had not received previous treatment responded to fludarabine (IC₅₀=0.56) but not gemcitabine or CHK1 inhibitor. Antagonistic effects were not seen in the fludarabine and CHK1 inhibitor combinations. In contrast, tumor cells isolated from a relapsed patient who one week prior received rituximab treatment, responded to the CHK1 inhibitor (Formula 1.1) (IC50=11 nM) but not to fludarabine (IC50=5.1 μM) or gemcitabine. Antagonistic effects were not seen in the combination groups.

### Example 2

### Demonstration of Increased Phospho-CHK1 in CLL Patient Samples that are Responsive to CHK1 Inhibition

Blood samples (10 mls) were collected from CLL patients and placed on ice. Whole blood was then diluted with PBS (2-3 fold dilution) and carefully layered over 12 mls Ficoll in a 50 ml conical tube. Samples were then centrifuged at 3500 rpm for 35 minutes without break at room temperature. Plasma was removed and opaque interphase containing mononuclear cells was collected and placed into a clean conical centrifuge tube. Cells were then washed twice with PBS with 1% FBS. Following final wash, cells are resuspended in 5˜10 mls RPMI 1640 plus 10% FCS medium. Cells are then counted by Trypan blue exclusion method and viability and purity assessed.

Cells are then plated into 12 well plates and incubated overnight in drug free medium. Isolated cells are then treated with vehicle, 0.1 μM gemcitabine, 3 and 10 μM fludarabine, 0.1, 0.25 and 0.5 μM CHK1 inhibitor (Formula 1.1) and combinations of gemcitabine or fludarabine with CHK1 inhibitor at the described doses. Following a 5 hour treatment, cells are harvested in Phosphosafe extraction reagent (Novagen, San Diego, Calif.)). Lysates are then prepared and protein concentrations determined by BCA protein assay kit (Pierce, Rockford, Ill.). ELISA assays are then performed as follows to determine phospho-CHK1 levels. In brief, total CHK1 antibody (Abcam, Cambridge, Mass.) is added to ELISA plates and incubated overnight at 4° C. Plates are then washed 3 times with PBST (phosphate buffered saline with 0.2% Tween20) and blocked with PBST containing 3% BSA for 3 hours. Experimental CLL cell lysate (4 ug) or controls and a dilution series of a standard lysate (standard curve) is then added and incubated for 2 hours. Plates are then washed 3 times as described above and anti-phospho-CHK1 antibody incubated for 3 hours at room temperature. Plates are then washed as above and incubated with Lance Eu anti-rabbit antibody (Perkin Elmer Life Science, Boston, Mass.) for 1 hour at room temperature. Plates are then washed as above, incubated with DELPFIA enhancement solution ((Perkin Elmer Life Science, Boston, Mass.) for 5 minutes at room temperature while shaking and read on a Victor² (Perkin Elmer Life and Analytical Science, Shelton, Conn.) using Europium program with time resolved fluorometry, 340 nm excitation and 615 nm emission with 400 μs delay, 400 μs window and 1000 μs cycle. Data is then converted to relative amounts based on the standard curve. The samples from patients who did not respond to CHK1 inhibitor had undetectable levels of phospho-CHK1 and the sample from a patient who did respond is found to have levels of phospho-CHK1 significantly above background (p>0.02).

### Example 3

### Demonstration of Single Agent Sensitivity of AML Patient Samples to a CHK1 Inhibitor

Marrow mononuclear cells from 9 patients with acute myelogenous leukemia were isolated by sedimentation on Ficoll-Hypaque density gradients (English et al. Single-Step Separation of Red Blood Cells. Granulocytes and Mononuclear Leukocytes on Discontinuous Density Gradients of Ficoll-Hypaque. Journal of Immunological Methods, 5: 249-252, 1974). After removal of an aliquot for staining to determine the percentage of blasts, replicate aliquots containing 1×10⁶ cells in 1 ml Improved Dulbecco's Medium containing 20% heat-inactivated fetal bovine serum, 50 units/ml penicillin G, 50 μg/ml streptomycin and 2 mM glutamine (medium A) were treated with diluent (0.2% dimethyl sulfoxide), increasing concentrations of cytarabine alone (25-300 nM), increasing concentrations of a CHK-1 inhibitor (formula 1.1) (30-300 nM), or increasing concentrations of cytarabine in the presence of 100 nM of a CHK-1 inhibitor (Formula 1.1). Following a 48-hour incubation at 37° C. in a humidified atmosphere containing 5% (v/v) CO₂, cells are sedimented at 100×g for 5 min, washed and resuspended in drug-free medium A. Duplicate aliquots containing 3×10⁵ cells were then mixed with 1.5 ml methylcellulose medium containing stem cell factor, granulocyte/macrophage colony-stimulating factor, granulocyte colony-stimulating factor, erythropoietin, interleukin-3 and interleukin-6 and plated in gridded 35-mm culture dishes. Following a 14-day incubation at 37° C., colonies containing ≧50 cells were scored on an inverted microscope using established criteria (Eaves, C. and Lambie, K. Atlas of human hematopoietic colonies. Vancouver: StemCell Technologies Inc., 1995).

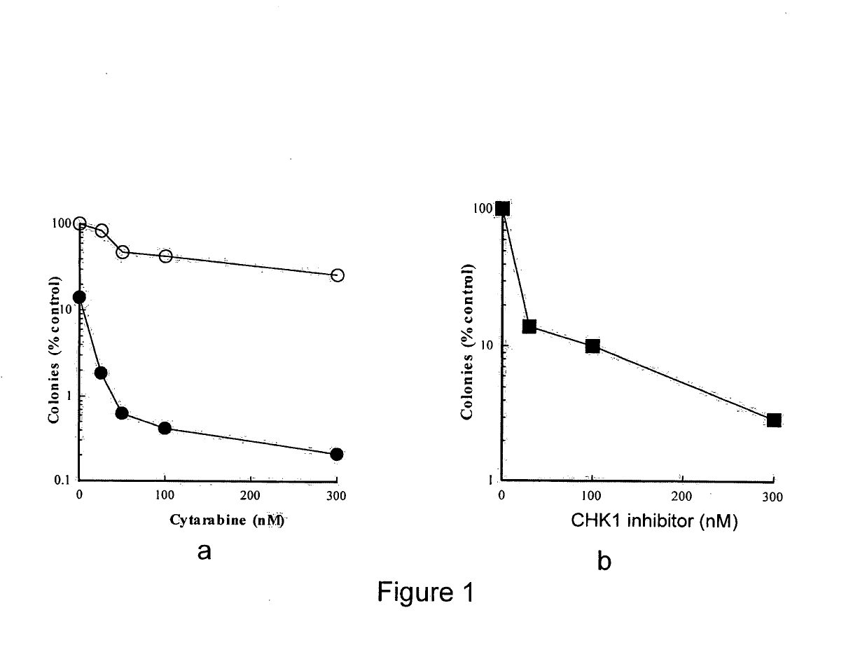

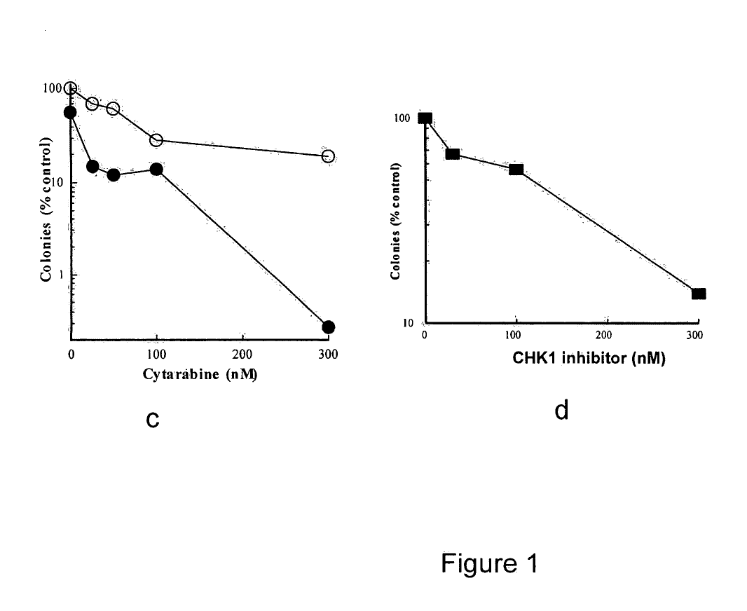

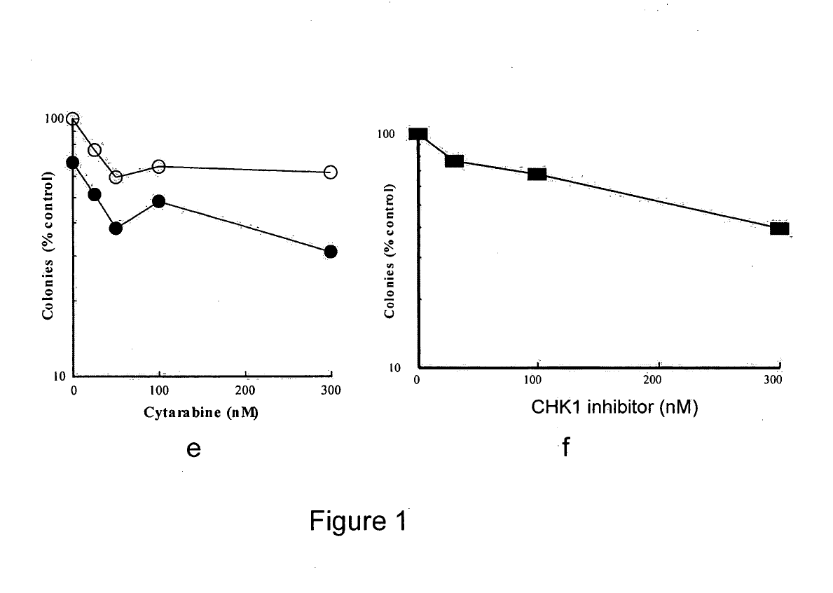

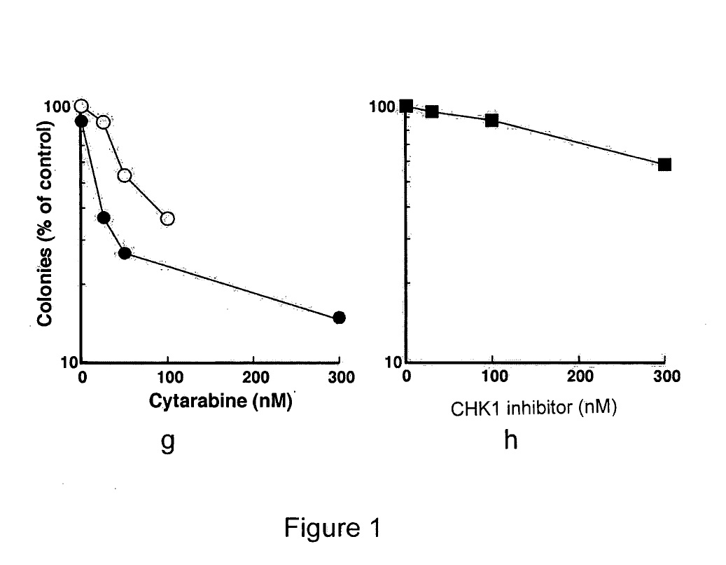

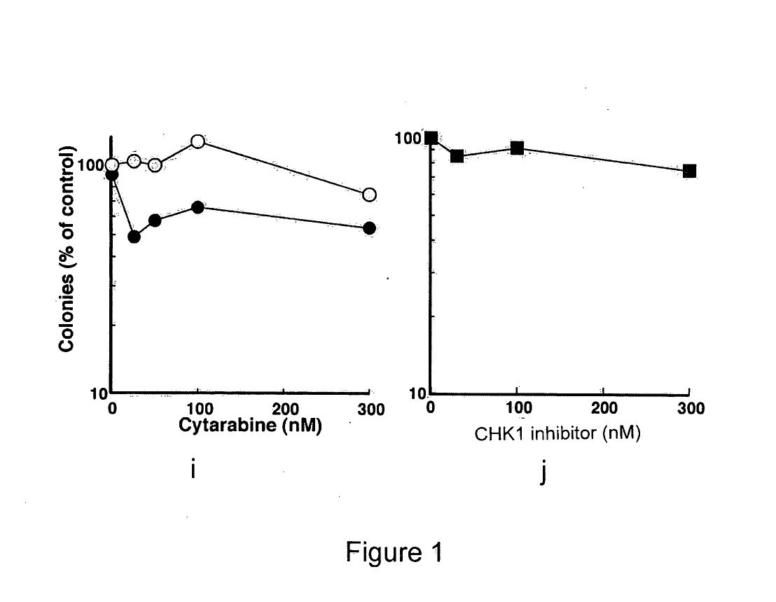

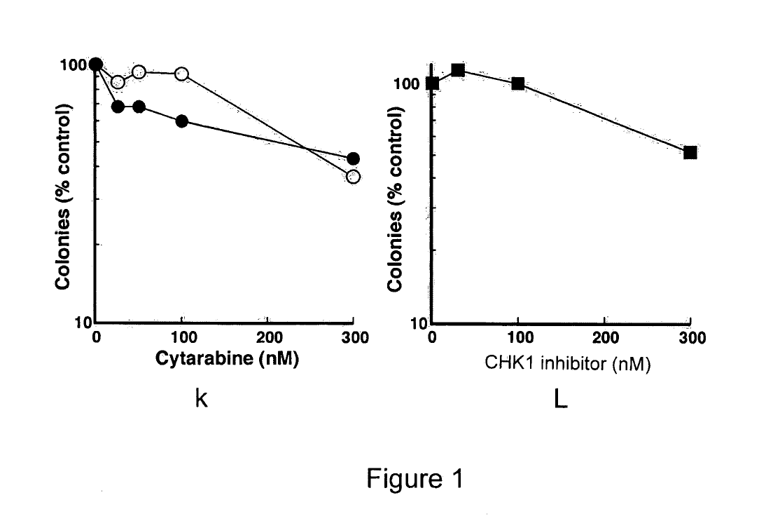

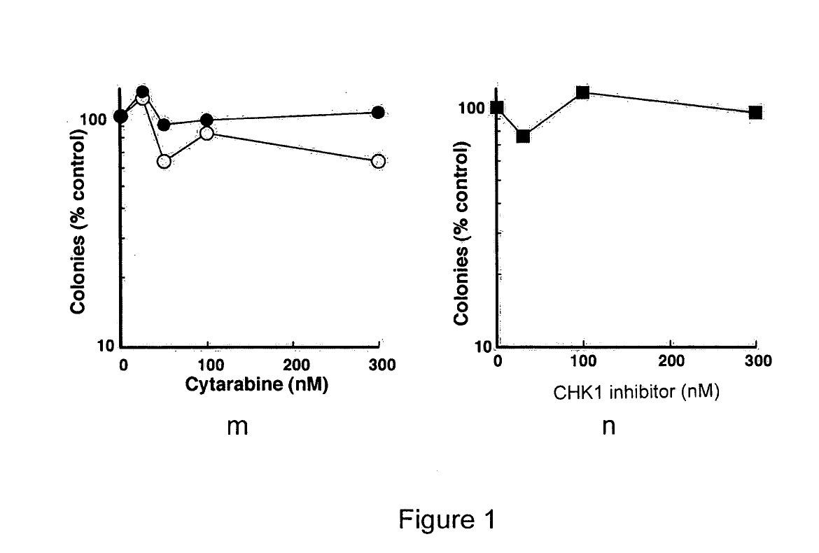

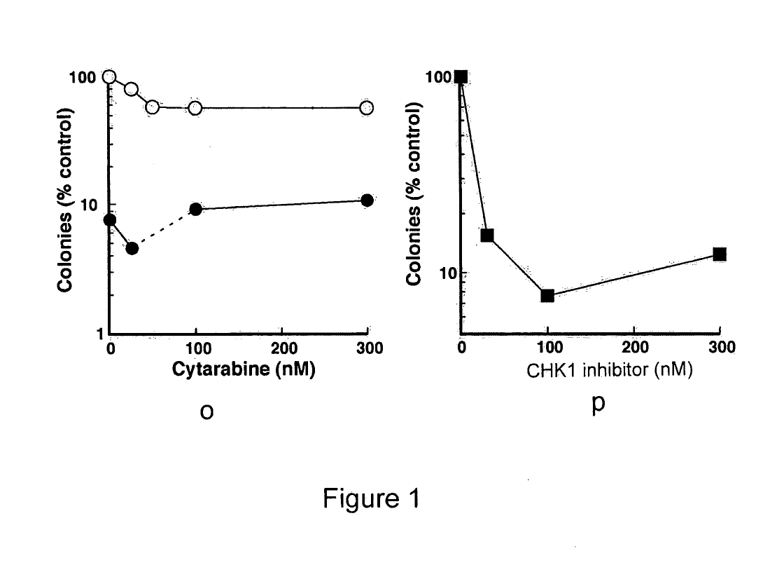

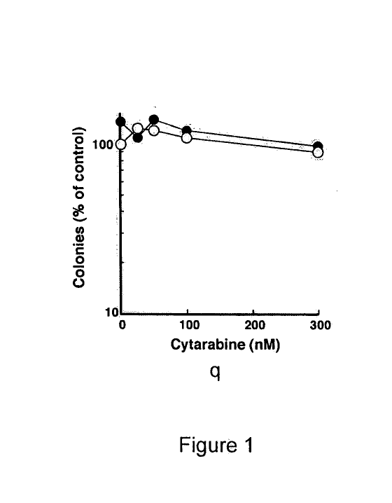

Results are shown in FIGS. 1(a-q). Line graphs with (▪) are results from AML patient samples treated with CHK1 inhibitor; line graphs with () are results from AML patient samples treated with CHK1 inhibitor and cytarabine; and line graphs with (◯) are results from AML patient samples treated cytarabine. The results from patient sample 1 are shown in FIGS. 1a and 1b; the results from patient sample 2 are shown in FIGS. 1c and 1d; the results from patient sample 3 are shown in FIGS. 1e and 1f; the results from patient sample 4 are shown in FIGS. 1g and 1h; the results from patient sample 5 are shown in FIGS. 1i and 1j; the results from patient sample 6 are shown in FIGS. 1k and 1l; the results from patient sample 7 are shown in FIGS. 1m and 1n (very low colony counts); the results from patient sample 8 are shown in FIGS. 1o and 1p (controls had low cell counts); and the results from patient sample 9 are shown in FIG. 1q. Four out of nine samples (1b, 1d, 1f and 1o) showed CHK1 single agent sensitivity.

1. A method to treat a patient suffering from a hematologic malignancy comprising administering to said patient a therapeutically effective amount of a CHK1 inhibitor simultaneously with or consecutively with a B cell depleting antibody.

2. The method of claim 1 wherein the hematologic malignancy is chronic lymphocytic leukemia.

3. The method of claim 1 wherein the B cell depleting antibody is an anti-CD20 antibody.

4. The method of claim 1 wherein said anti-CD20 antibody is rituximab.

5. A method to treat a patient suffering from a B cell related tumor comprising administering to said patient a therapeutically effective amount of a CHK1 inhibitor simultaneously with or consecutively with a B cell depleting antibody.

6. The method of claim 5 wherein the B cell depleting antibody is an anti-CD20 antibody.

7. The method of claim 5 wherein said anti-CD20 antibody is rituximab.

8. A method to treat a patient suffering from a hematologic malignancy that is resistant to treatment with a DNA damaging agent comprising administering to said patient a therapeutically effective amount of a CHK1 inhibitor simultaneously with or consecutively with a B cell depleting antibody.

9. The method of claim 8 wherein the hematologic malignancy is chronic lymphocytic leukemia.

10. The method of claim 8 wherein the B cell depleting antibody is an anti-CD20 antibody.

11. The method of claim 10 wherein said anti-CD20 antibody is rituximab.

12. The method of claim 8 wherein said DNA damaging agent is fludarabine.

13. A method to treat a patient suffering from a B cell related tumor that is resistant to treatment with a DNA damaging agent comprising administering to said patient a therapeutically effective amount of a CHK1 inhibitor simultaneously with or consecutively with a B cell depleting antibody.

14. A method to treat a patient suffering from a B cell related tumor that is resistant to treatment with a DNA damaging agent comprising administering to said patient a therapeutically effective amount of a CHK1 inhibitor.

15. The method of claim 13 wherein the B cell depleting antibody is an anti-CD20 antibody.

16. The method of claim 13 wherein said anti-CD20 antibody is rituximab.

17. The method of claim 13 wherein said DNA damaging agent is fludarabine.

18. The method of claim 1 wherein the CHK1 inhibitor is a compound selected from the group consisting of Formulae 1.1-1.52, in free form or in a pharmaceutically acceptable salt form or in the form of a pharmaceutically acceptable solvate of the compound or the salt thereof.

19. The method of claim 1 wherein the CHK1 inhibitor is a compound of Formula 1.1 or Formula 1.2, in free form or in a pharmaceutically acceptable salt form or in the form of a pharmaceutically acceptable solvate of the compound or the salt thereof.

20.-30. (canceled)
